(19) United States
(12) Patent Application Publication
(10) Pub. No.: US 2025/0095856 A1
(43) Pub. Date: Mar. 20, 2025

(54) EMOTION ESTIMATION DEVICE

(71) Applicant: YAMAHA HATSUDOKI KABUSHIKI KAISHA, Iwata-shi (JP)

(72) Inventors: Keisuke SHIMA, Yokohama-shi (JP); Tsubasa NISHIHARA, Yokohama-shi (JP); Prasetia UTAMA PUTRA, Yokohama-shi (JP); Akikatsu KAMIYA, Sapporo-shi (JP); Shigenobu MINAMI, Sapporo-shi (JP); Shinichi INOUE, Shizuoka (JP); Yoshikazu KOIKE, Shizuoka (JP); Akira SAMESHIMA, Shizuoka (JP)

(73) Assignee: YAMAHA HATSUDOKI KABUSHIKI KAISHA, Iwata-shi (JP)

(21) Appl. No.: 18/967,905

(22) Filed: Dec. 4, 2024

Related U.S. Application Data
(63) Continuation-in-part of application No. PCT/JP2022/022695, filed on Jun. 4, 2022.

(51) Int. Cl. G16H 50/20

(52) U.S. Cl. CPC G16H 50/20

(57) ABSTRACT

An emotion estimation device including a storage device and a controller. The controller acquires the biological information of a user and store the biological information in the storage device, obtain an emotion estimation model used for estimation an emotion based on the biological information, and estimate the emotion of the user using the emotion estimation model. The emotion estimation model includes a total number m of models that are: a primary model that is used for estimating the emotion based on a plurality of types of the biological information and generates first-order knowledge information, and a nth-order-biological-information-distillation model that generates a nth-order knowledge information, 2<=n<=m, the nth-order-biological-information-distillation model being created based on a (n−1)th-order knowledge information. The controller estimates the emotion of the user using the nth-order-biological-information-distillation model, based on a subset of the plurality of types of the biological information, and outputs the information related to the estimated emotion as the emotion information.

### CROSS-REFERENCES TO RELATED APPLICATIONS

This is a continuation-in-part application of International Application No. PCT/JP2022/022695 filed on Jun. 4, 2022, the contents of which are incorporated herein by reference.

### TECHNICAL FIELD

The present teaching relates to an emotion estimation device.

### BACKGROUND ART

An emotion of a person affects action, thought, and physical condition of the person in some cases. Also, action, thought, and physical reaction of a person affect the emotion of the person in some cases. For example, behavior, way of thinking, physical condition, or the like of a person change when the person feels joy, sad, angry, hatred, fear, surprised, or the like. Moreover, an emotion of a person changes when the person moves his or her body, feels fatigue, drowsiness, and pain, receives an external stimulus, and recalls a memory, or the like. As described above, an emotion and a physical condition are related to each other.

An emotion, such as joy, sadness, anger, hatred, fear, or surprise, is a mental activity, and therefore, it is difficult to directly recognize it. On the other hand, the emotion is related to a physical condition. Therefore, there has been a known technology that estimates an emotion of a person, based on change in mental and physical conditions of the person. Patent Document 1 discloses an emotion analysis method for analyzing an emotion of a test subject, based on a heart rate signal that is one type of biological information expressing mental and physical conditions of the test subject, and an emotion analysis device thereof. The emotion analysis device analyzes the acquired heart rate signal of the test subject and calculates features, such as an R-R interval, a low-frequency range power spectrum, a high-frequency range power spectrum, a power spectral ratio, or the like. Furthermore, the emotion analysis device estimates an emotion with respect to the heart rate signal, based on the features, using an emotion analysis model created by learning.

A heart rate signal, such as an electrocardiogram signal, or a pulse wave, that are biological information of a person can be easily measured by a wearable sensor, such as a wristband, that can be easily attached. The heart rate signal is measured in real time under various situations by attaching the wearable sensor. Accordingly, the emotion analysis device of Patent Document 1 using a heart rate signal that can be easily measured increases convenience in estimating an emotion.

### CITATION LIST

### Patent Document

Patent Document 1: European Patent Application Laid-Open Publication No. 3263024

### SUMMARY OF INVENTION

### Technical Problem

The emotion analysis model that estimates an emotion with respect to the heart rate signal can increase emotion estimation accuracy by learning using training data in which heart rate signals of various test subjects in various patterns and emotions of the test subjects are combined. However, when an emotion is analyzed using the training data including a combination of one type of biological information and at least one emotion, a difference in the biological information due to a difference in the emotion is less clear. Therefore, when learning is performed using the training data including only one type of biological information, the emotion analysis model needs to perform learning using a large amount of the training data. Moreover, many test subjects and a lot of time and efforts are required for measuring the heart rate signal for creating the large amount of the training data. For this reason, in the emotion analysis model that estimates an emotion of a user, based on the one type of biological information, it is difficult to increase emotion estimation accuracy.

On the other hand, when an emotion is analyzed using the training data including combinations of a plurality of types of biological information including electrocardiogram signal, pulse wave, brain wave, sweating, or exhalation, and at least one emotion, a difference in the biological information due to a difference in emotion tends to be clear. Therefore, when learning is performed using the training data including the plurality of types of the biological information, the emotion analysis model can have an increased emotion estimation accuracy by a smaller amount of the training data than that of the training data used in performing learning using the training data including only one type of biological information.

However, when the emotion of the user is estimated based on the plurality of types of biological information, the emotion analysis device needs to acquire the plurality of types of biological information from the user at a time. Therefore, convenience of the emotion analysis model that estimates the emotion of the user, based on the plurality of types of biological information, is reduced as the types of biological information that are acquired are increased. Therefore, an emotion estimation device that can highly accurately estimate an emotion while increasing convenience in acquiring biological information in estimating the emotion is desired.

It is therefore an object of the present teaching to provide an emotion estimation device that can highly accurately estimate an emotion while increasing convenience in biological information acquisition in estimating the emotion.

### Solution to Problem

The inventors of the present teaching have reached a configuration of an emotion estimation device that can highly accurately estimate an emotion while increasing convenience in biological information acquisition in estimating the emotion.

An emotion estimation device for estimating an emotion of a user, according to one embodiment of the present teaching includes a storage device that stores biological information of the user, and a controller including a processor, the controller being configured to: acquire the biological information of the user to thereby store the biological information in the storage device, the biological information including a plurality of types, obtain an emotion estimation model used for estimating an emotion based on the biological information, estimate the emotion of the user, based on the biological information of the user stored in the storage device, using the emotion estimation model, and outputs information related to the estimated emotion as emotion information.

Herein, the emotion estimation model includes a total number m of models, m being an integer of 2 or larger, that are: a primary model, which is used for estimating the emotion based on the plurality of types of the biological information, and generates first-order knowledge information, and a nth-order-biological-information-distillation model that generates a nth-order knowledge information, n being each integer between 2 and m, the nth-order-biological-information-distillation model being created based on a (n−1)th-order knowledge information.

The controller estimates the emotion of the user using the nth-order-biological-information-distillation model, based on a subset of the plurality of types of the biological information of the user that are used in the emotion estimation by the primary model, and outputs the information related to the estimated emotion as the emotion information.

In the above-described configuration, the emotion estimation model includes the nth-order-biological-information-distillation model created using the (n−1)th-order-knowledge information obtained from the (n−1)th-order-biological-information-distillation model. The (n−1)th-order-knowledge information inherits information used for estimating the emotion based on the biological information of the user, obtained by the primary model used for estimating the emotion based on the plurality of types of the biological information by learning based on the plurality of types of the biological information. That is, by using the (n−1)th-order-knowledge-information, the nth-order-biological-information-distillation model inherits privileged information that is information absent from the biological information used for learning. Therefore, even when being used for estimating the emotion of the user, based on the biological information that is included in the types of the biological information necessary for estimating the emotion of the user using the emotion estimation model and the number of the types of which is smaller than the number of the types of the biological information necessary for estimating the emotion of the user using the primary model, the controller can achieve estimation accuracy equal to emotion estimation accuracy of the primary model in some cases. Thus, by using the biological information that can be easily acquired from the user for estimating the emotion, it is possible to increase emotion estimation accuracy while increasing convenience in biological information acquisition in estimating the emotion of the user.

According to another aspect, the emotion estimation device of the present teaching preferably has the following configuration. The nth-order-biological-information-distillation model is created using the first-order knowledge information obtained by the primary model created based on the plurality of types of the biological information, the biological information including first biological information that is at least one of the biological information that is independent of a surrounding environment of the user, a work of the user, or an action of the user, the biological information that can be easily acquired from the user, or the biological information that is independent of the surrounding environment of the user, the work of the user, or the action of the user and can be easily acquired from the user, or using the (n−1)th-order-knowledge information obtained by the (n−1)th-order-biological-information-distillation model created based on the biological information including the first biological information. The controller estimates the emotion of the user, based on the first biological information, using the nth-order-biological-information-distillation model.

As described above, the nth-order-biological-information-distillation model is used for estimating the emotion, based on the plurality of types of the biological information including the first biological information. Therefore, the controller can estimate the emotion of the user based on the biological information including the biological information that can be easily acquired in a relatively stable state among the plurality of types of the biological information of the user using the nth-order-biological-information-distillation model. The emotion estimation accuracy in the nth-order-biological-information-distillation model can be further increased by using a plurality of types of biological information of the first biological information. Thus, by using the biological information that can be easily acquired from the user for estimating the emotion of the user, emotion estimation accuracy can be increased while increasing convenience in biological information acquisition in estimating the emotion of the user.

According to another aspect, the emotion estimation device of the present teaching preferably has the following configuration. The nth-order-biological-information-distillation model is created using the first-order knowledge information obtained by the primary model created based on the plurality of types of the biological information, the biological information including second biological information that is at least one of the biological information that is dependent on a surrounding environment of the user, a work of the user, or an action of the user, the biological information that is difficult to acquire easily from the user, or the biological information that is dependent on the surrounding environment of the user, the work of the user or the action of the user and is difficult to acquire easily from the user, or using the (n−1)th-order-knowledge information obtained by the (n−1)th-order-biological-information-distillation model created based on the biological information including the second biological information. The controller estimates the emotion of the user, based on biological information of the user that is free of the second biological information, using the nth-order-biological-information-distillation model.

As described above, the nth-order-biological-information-distillation model is created using the knowledge information obtained by the primary model or the (n−1)th-order-biological-information-distillation model, each of the primary model and the (n−1)th-order-biological-information-distillation model being created based on the plurality of types of the biological information including the second biological information. On the other hand, the controller estimations the emotion of the user, based on the biological information that is free of the second biological information among the plurality of types of the biological information of the user and that includes the biological information that can be easily acquired in a relatively stable state, using the nth-order-biological-information-distillation model. That is, by using the nth-order-biological-information-distillation model that inherits the knowledge information obtained by the primary model or the (n−1)th-order-biological-information-distillation model, the controller can estimate the emotion of the user, based on the knowledge information created based on the second biological information, and the biological information that is free of the second biological information. Thus, emotion estimation accuracy can be increased while increasing convenience in biological information acquisition in estimating the emotion of the user.

According to another aspect, the emotion estimation device of the present teaching preferably has the following configuration. The nth-order-biological-information-distillation model is created using the first-order knowledge information obtained by the primary model created based on the plurality of types of the biological information, the biological information including third biological information that is at least two of a brain wave, sweating, respiration, a pulse wave, or an electrocardiogram signal, or using the (n−1)th-order-knowledge information obtained by the (n−1)th-order-biological-information-distillation model created based on the biological information including the third biological information. The controller estimates the emotion of the user, based on the biological information, using the nth-order-biological-information-distillation model, the biological information including a subset of types of the biological information included in the third biological information and a portion of each biological information constituting the subset.

As described above, the nth-order-biological-information-distillation model is created using the knowledge information obtained by the primary model or the (n−1)th-order-biological-information-distillation model created based on the plurality of types of the biological information including the third biological information. On the other hand, the nth-order-biological-information-distillation model is used for estimating the emotion, based on the biological information that includes the plurality of types of the biological information included in the third biological information, the number of types of which is smaller than the third biological information, and a portion of each of the plurality of types of the biological information. That is, by using the nth-order-biological-information-distillation model that inherits the knowledge information obtained by the primary model or the (n−1)th-order-biological-information-distillation model, the controller can estimate the emotion of the user based on the knowledge information created based on the third biological information, and the plurality of types of the biological information the number of types of which is smaller than the number of the types of the third biological information. Thus, emotion estimation accuracy can be increased while increasing convenience in biological information acquisition in estimating the emotion of the user.

According to another aspect, the emotion estimation device of the present teaching preferably has the following configuration. Among biological information related to a brain wave, sweating, respiration, an electrocardiogram signal, or a pulse wave of the user, the controller estimates the emotion of the user based on the biological information related to at least one of the electrocardiogram signal, the respiration, or the pulse wave using the emotion estimation model.

As described above, by using the emotion estimation model including the nth-order-biological-information-distillation model that inherits the (n−1)th-order-knowledge information, the controller can estimate the emotion of the user, based on the biological information related to at least one of the types of the biological information related to the electrocardiogram signal, the respiration, or the pulse wave of the user that can be relatively easily measured among the biological information related to the brain wave, the sweating, the respiration, the pulse wave, or the electrocardiogram signal of the user. Moreover, emotion estimation accuracy in the emotion estimation device can be further increased in some cases by using a plurality of types of biological information among the biological information related to the electrocardiogram signal, the respiration, or the pulse wave of the user. Thus, by using the biological information that can be easily acquired from the user for estimating the emotion, it is possible to increase emotion estimation accuracy while increasing convenience in biological information acquisition in estimating the emotion of the user.

According to another aspect, the emotion estimation device of the present teaching preferably has the following configuration. The (n−1)th-order-biological-information-distillation model includes a plurality of internal models used for estimating the emotion, based on the plurality of types of the biological information. The nth-order-biological-information-distillation model is created using the (n−1)th-order-knowledge information obtained based on the plurality of types of the biological information, using the plurality of internal models.

As described above, in the (n−1)th-order-biological-information-distillation model including the plurality of internal models, error and variation of an emotion estimated based on the biological information of the user are suppressed by ensemble learning in which respective outputs of the internal models are averaged, or ensemble learning in which, considering a parameter of one of the internal models, parameters of the other ones of the internal models are improved, or the like. Therefore, the nth-order-biological-information-distillation model that has performed learning using the (n−1)th-order-knowledge information obtained by the (n−1)th-biological-information-distillation model can suppress variation of emotion estimation accuracy of estimation of the emotion of the user based on the biological information of the user. Thus, by using the biological information that can be easily acquired from the user for estimating the emotion, it is possible to increase emotion estimation accuracy while increasing convenience in biological information acquisition in estimating the emotion of the user.

According to another aspect, the emotion estimation device of the present teaching preferably has the following configuration. The emotion estimation model includes the nth-order-biological-information-distillation model created by learning the (n−1)th-order-knowledge information that is obtained using the (n−1)th-order-biological-information-distillation model based on, among biological information related to a brain wave, sweating, respiration, a pulse wave or an electrocardiogram signal, a plurality of types of the biological information including the biological information related to at least one of the electrocardiogram signal, the respiration or the pulse wave, and the biological information including the biological information related to at least one of the electrocardiogram signal, the respiration or the pulse wave.

As described above, the nth-order-biological-information-distillation model is created using the (n−1)th-order-knowledge information and the biological information related to at least one of the electrocardiogram signal, the respiration, or the pulse wave. Therefore, the controller can estimate the emotion of the user, based on at least one of the types of biological information related to the electrocardiogram signal, the respiration, or the pulse wave that can be easily measured, using the nth-order-biological-information-distillation model. Thus, emotion estimation accuracy can be increased while increasing convenience in biological information acquisition in estimating the emotion of the user.

According to another aspect, the emotion estimation device of the present teaching preferably has the following configuration. Each of the nth-order-biological-information-distillation model and the (n−1)th-order-biological-information-distillation model is a neural network model.

As described above, the nth-order-biological-information-distillation model is a neural network model created using the (n−1)th-order-biological-information model that is a neural network model including an intermediate layer. In the nth-order-biological-information-distillation model, parameters are increased by the intermediate layer, emotion estimation accuracy is increased in accordance with a number of the intermediate layers. Thus, by using a plurality of types of the biological information that can be easily acquired from the user for estimating the emotion, it is possible to increase emotion estimation accuracy while increasing convenience in biological information acquisition in estimating the emotion of the user.

According to another aspect, a portable terminal according to the present teaching preferably has the following configuration. The portable terminal is a portable terminal that acquires biological information of a user used for estimating the emotion of the user in the emotion estimation device of the present teaching. The portable terminal includes a portable terminal memory that stores the acquired biological information of the user, and a portable terminal processor. The portable terminal processor acquires the biological information of the user to store the biological information in the portable terminal memory and outputs the biological information of the user stored in the portable terminal memory.

As described above, the emotion estimation device acquires the biological information of the user used for estimating the emotion of the user by the portable terminal. The portable terminal outputs the acquired biological information of the user to the controller of the emotion estimation device located outside the portable terminal processor. Therefore, the portable terminal having the above-described function is a portable terminal used only for production of the emotion estimation device. The portable terminal can increase convenience in biological information acquisition in estimating the emotion of the user.

According to another aspect, a portable terminal according to the present teaching preferably has the following configuration. The portable terminal is configured to be communicable with outside. The portable terminal processor outputs the biological information of the user stored in the portable terminal memory to the emotion estimation device that is outside the portable terminal.

As described above, the portable terminal that has a communication function and the emotion estimation device constitute an emotion estimation system including a terminal that acquires the biological information and a server that estimates the emotion, based on the biological information. Therefore, the portable terminal that acquires the biological information of the user and outputs the biological information to the emotion estimation device via communication is a portable terminal used only for production of the emotion estimation system. As described above, by acquiring the biological information of the user using the communication function of the portable terminal, it is possible to increase convenience in biological information acquisition in estimating the emotion of the user.

According to another aspect, an emotion estimation device according to the present teaching preferably has the following configuration. The emotion estimation device is configured to be communicable with outside. The controller acquires the biological information from a portable terminal.

As described above, the portable terminal that has a communication function and the emotion estimation device constitute an emotion estimation system including a terminal that acquires the biological information and a server that estimates the emotion, based on the biological information. Therefore, the emotion estimation device that acquires the biological information of the user from the portable terminal and performs emotion estimation, based on the biological information, is an emotion estimation device used for only production of the emotion estimation system. As described above, by acquiring the biological information of the user using the communication function of the portable terminal, it is possible to increase convenience in biological information acquisition in estimating the emotion of the user.

According to another aspect, an emotion estimation device according to the present teaching preferably has the following configuration. The emotion estimation device is a portable terminal.

As described above, the portable terminal acquires the biological information of the user output from the biological information acquirer and estimates the emotion of the user using the emotion estimation model by the portable terminal processor. As described above, by using a function of the portable terminal owned by the user to acquire the biological information of the user and estimate the emotion of the user, it is possible to increase emotion estimation accuracy while increasing convenience in biological information acquisition in estimating the emotion of the user.

According to another aspect, a program that controls the emotion estimation device of the present teaching preferably has the following configuration. The program causes the storage device to store the biological information of the user. The program causes the controller to estimate the emotion of the user, using the nth-order-biological-information-distillation model, based on the biological information of the user that is included in the types of the biological information necessary for estimating the emotion using the primary model and the number of types of which is smaller than the number of the types of the biological information necessary for estimating the emotion using the primary model. The program outputs information related to the estimated emotion as the emotion information.

As described above, the program causes the storage device to operate such that the storage device of the emotion estimation device stores the biological information of the user, and causes the controller to operate such that the controller estimates the emotion of the user, based on the biological information, using the nth-order-biological-information-distillation model. Therefore, the program is a program used for production of the emotion estimation device and is an essential program for solving a problem of highly accurately estimating an emotion while increasing convenience in biological information acquisition in estimating the emotion. The emotion estimation device can be produced by installing the program, and the emotion estimation device can be transferred by downloading the program.

The terminology used herein is for the purpose of describing particular embodiments only and is not intended to be limiting of the teaching.

As used herein, the term “and/or” includes any and all combinations of one or more of the associated listed items.

It will be further understood that the terms “including,” “comprising” or “having” and variations thereof when used in this specification, specify the presence of stated features, steps, operations, elements, components, and/or their equivalents, but do not preclude the presence or addition of one or more steps, operations, elements, components, and/or groups thereof.

It will be further understood that the terms “mounted,” “connected,” “coupled,” and/or their equivalents thereof are used broadly and encompass both “direct and indirect” mounting, connecting, and coupling. Furthermore, “connected” and “coupled” are not restricted to physical or mechanical connections or couplings, and can include electrical connections or couplings whether direct or indirect.

Unless otherwise defined, all terms (including technical and scientific terms) used herein have the same meaning as commonly understood by one having ordinary skill in the art to which this teaching belongs.

It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the present teaching, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the present teaching.

In this specification, embodiments of an emotion estimation device according to the present teaching will be described.

In the following description, numerous specific details are set forth in order to provide a thorough understanding of the present teaching. It will be evident, however, to one skilled in the art that the present teaching can be practiced without these specific details.

Therefore, the following disclosure is to be considered as an exemplification of the present teaching, and is not intended to limit the present teaching to the specific embodiments illustrated by the figures or description below.

### [User]

As used herein, the term “user” refers to a person whose emotion is estimated by an emotion estimation device. Biological information is acquired from the user by the emotion estimation device. An emotion of the user is estimated by the emotion estimation device, based on the acquired biological information.

### [Information Related to User]

As used herein, the term “information related to a user” includes information related to the user, such as sex, age, physical features such as height or weight, personality, likes, or taste of the user. The information related to the user is input to the emotion estimation device by the user himself or herself in advance. The information related to the user is free of personal information, such as name or address, that identifies the user.

### [External Event]

As used herein, the term “external event” includes an event, a phenomenon, and a situation that provide a user with information, stimulus, or the like from outside, such as a surrounding environment in which the user is located, or a work that the user is performing. The external event is, for example, a state where the user is moving by a leaning vehicle, a state where the user is moving on foot, a state where the user is looking at a photo of an animal, or the like.

### [Test Subject]

As used herein, the term “test subject” refers to a person whose biological information for training data used for learning of an emotion estimation model used in emotion estimation is measured. The biological information of the test subject with respect to a particular external event is measured and the test subject provides emotion information that is information related to the emotion, based on self-assessment. The test subject provides the emotion information, based on self-assessment, for each biological information with respect to the external event.

### [Learning]

As used herein, the term “learning” or “machine learning” refers to a work in which a parameter of a model (program) is adjusted such that an output with respect to an input is a correct answer in a machine (processor). In an embodiment, of supervised learning in which learning is performed based on training data in which input data and a correct answer to the input data are combined and unsupervised learning in which determination of correctness is made using only input data, at least the supervised learning is performed on a primary model, a secondary-biological-information-distillation model, and a tertiary-biological-information-distillation model that are learning targets.

### [Biological Information]

As used herein, the term “biological information” refers to various types of physiological and anatomical information sent by a user or a test subject. The biological information refers to all of life activities that occur in a living body of the user or the test subject. The biological information includes information related to, for example, heart rate, heat sound, heart rate waveform, cardiac cycle, heart rate change, blood pressure, pulse wave, triaxial acceleration, body surface temperature, brain wave, respiratory rate, pupil state, myoelectric potential, blood component, exhalation, exhalation amount, exhalation component, or the like of the user. The biological information is measured by a heart rate sensor, an acceleration sensor, a temperature sensor, or the like that is a wearable sensor worn by the user.

### [Biological Information for Learning]

As used herein, the term “biological information for learning” refers to biological information of a test subject used for learning of the primary model, the secondary-biological-information-distillation model, and the tertiary-biological-information-distillation model. The biological information for learning includes information related to, for example, heart rate, heat sound, heart rate waveform, cardiac cycle, heart rate change, blood pressure, pulse wave, triaxial acceleration, body surface temperature, brain wave, respiratory rate, pupil state, myoelectric potential, blood component, exhalation, exhalation amount, exhalation component, or the like of the test subject. The biological information for learning is measured by a heart rate sensor, an acceleration sensor, a temperature sensor, or the like that is a wearable sensor worn by the test subject.

### [Biological Information Related to Heart Rate]

As used herein, the term “biological information related to heart rate” refers to information including heart rate, cardiac cycle, heart rate change, blood pressure, electrocardiogram signal, pulse wave, or the like of a user among biological information. The biological information related to heart rate is measured by a heart rate sensor provided to a wearable sensor or the like that is a biological information acquiring section worn by the user.

### [Emotion Information]

As used herein, the term “emotion information” refers to information expressing a state where a user feels joy, anger, grief, pleasure, or the like. The emotion information related to the emotions includes a state where the user feels happy, a state where the user feels relaxed, a state where the user feels angry, and a state where the user feels sad. The information related to the emotions includes information related to a probability of an emotion of the user estimated based on the acquired biological information. The emotion information may include information related to a degree of an emotion that the user feels. One type of the emotion information may include a plurality of emotions. The emotion information is created by a controller provided to, for example, a portable terminal held by the user, or a server that acquired the biological information.

### [Degree]

As used herein, the term “degree” is an expression expressing a strength of an emotion in a stepwise manner. For example, a degree of comfortableness is obtained by dividing comfortableness into a plurality of stages from a standard emotion with which an emotion related to comfortableness is dominant and the emotion related to comfortableness appears most strongly to a standard emotion with which the emotion related to comfortableness is dominant and the emotion related to comfortableness is weakest (feeling neither uncomfortable nor comfortable), and allocating a strength of the standard emotion related to comfortableness to each stage.

### [Model]

As used herein, the term “model” refers to specific calculation formula, function, and calculation method obtained after learning has been performed. The model is a mathematical expression expressing a relationship of an output with respect to an input. The model converts input data in accordance with the mathematical expression. Creating a model refers to performing learning of the model. As the model, there are a regression model that predicts a value, a classification model that classifies data, or the like.

### [Neural Network Model]

As used herein, the term “neural network model” refers to a mathematical model in which a plurality of processing units that linearly transform inputs are combined with each other. The neural network model includes an input layer, at least one intermediate layer, an output layer, a function that combines the input layer and the intermediate layer, and a function that combines the intermediate layer and the output layer. The input layer, the intermediate layer, and the output layer are a plurality of variables to which data is input. The neural network model sequentially converts data input to the input layer by the functions and output the converted data. Parameters of the functions in the neural network model are adjusted (learned) such that data that is output when training data including a combination of data and a correct answer to the data is input to the input layer approaches the correct answer of the training data. An error between the data that is output based on the input data and the correct answer can be reduced by repeating learning of the neural network model.

### [Knowledge Information]

As used herein, the term “knowledge information” refers to information including at least part of knowledge that is an output of the intermediate layer of the model, an output of the output layer thereof, information of the intermediate layer, or information of the output layer.

### [Distillation of Knowledge]

As used herein, the term “distillation of knowledge” refers to a method in which knowledge information of a teacher model, which is a learning model that has learned based on training data, is used to enable another model, which is a student model, to learn. For example, distillation of knowledge is a learning method for the student model, based on a smaller number of types of training data than a number of types of training data used in learning of the teacher model, and the knowledge information created using the teacher model.

### [First Biological Information]

As used herein, the term “first biological information” refers to biological information related to at least one of the biological information that is less likely to be affected by a surrounding environment of a user, a work of the user, or an action of the user, the biological information that can be easily acquired from the user, or the biological information that is less likely to be affected by the surrounding environment of the user, the work of the user, or the action of the user and can be easily acquired from the user. The first biological information includes biological information related to, for example, at least one of electrocardiogram signal, respiration, pulse wave, brain wave, or sweating of the user. The first biological information includes biological information related to, for example, at least one of the electrocardiogram signal, the respiration, or the pulse wave of the user. The first biological information includes biological information related to, for example, a combination of the electrocardiogram signal, the respiration, and the pulse wave of the user, a combination of the electrocardiogram and the respiration of the user, a combination of the electrocardiogram signal and the pulse wave of the user, a combination of the respiration and the pulse wave of the user, or the electrocardiogram signal, the respiration, the pulse wave, the brain wave, or the sweating of the user.

### [Second Biological Information]

As used herein, the term “second biological information” refers to biological information related to at least one of biological information that is likely to be affected by an surrounding environment of a user, a work of the user, or an action of the user, biological information that is difficult to acquire easily from the user, or biological information that is likely to be affected by the surrounding environment of the user, the work of the user or the action of the user and is difficult to acquire easily from the user. The second biological information includes biological information related to, for example, at least one of electrocardiogram signal, respiration, pulse wave, brain wave, or sweating of the user. The second biological information includes biological information related to, for example, a combination of the brain wave and the sweating of the user, or the brain wave or the sweating of the user. For example, a device that detects the brain wave includes very many sensors, and therefore, the brain wave is difficult to acquire easily. For example, the sweating is likely to be affected by the surrounding environment of the user or greatly differs among individuals in some cases.

[Relationship between First Biological Information and Second Biological Information]

In this specification, the first biological information and the second biological information may be determined in accordance with a relative relationship of types of biological information. For example, the first biological information may be the pulse wave or the respiration of the user, and the second biological information may be the electrocardiogram signal or the brain wave of the user. For example, the first biological information may be the electrocardiogram signal of the user, and the second biological information may be the brain wave of the user. As described above, for example, the electrocardiogram signal of the user can be the first biological information and can be also the second biological information.

### [Third Biological Information]

As used herein, the term “third biological information” refers to biological information that is considered to be related to an emotion of a user. The third biological information includes biological information related to, for example, a combination of at least two of brain wave, sweating, respiration, pulse wave, or electrocardiogram signal that are five types of biological information, a combination of at least three of the brain wave, the sweating, the respiration, the pulse wave, or the electrocardiogram signal that are five types of biological information, a combination of at least four of the brain wave, the sweating, the respiration, the pulse wave, or the electrocardiogram signal that are five types of biological information, or a combination including the brain wave, the sweating, the respiration, the pulse wave, and the electrocardiogram signal that are five types of biological information.

### [Primary Model]

In this specification, a primary model may be a primary-biological-information-distillation model. In this case, the primary-biological-information-distillation model is a student model which is created using knowledge information of a teacher model used for emotion estimation based on a plurality of types of biological information and which is used for emotion estimation based on a plurality of types of biological information. For example, when a model used for emotion estimation based on a plurality of types of biological information is defined as a teacher model and a model created using the knowledge information of the teacher model and used for emotion estimation based on a plurality of types of biological information is defined as a student model, a primary model in the present teaching may be the teacher model described above, and may be the student model described above.

### Advantageous Effects of Invention

According to one embodiment of the present teaching, it is possible to increase emotion estimation accuracy while increasing convenience in biological information acquisition in estimating an emotion.

### BRIEF DESCRIPTION OF DRAWINGS

FIG. 1 is a diagram schematically illustrating learning on a secondary-biological-information-distillation model or a tertiary-biological-information-distillation model included in an emotion estimation model in an emotion estimation device according to a first embodiment of the present teaching and illustrating an overall configuration of the emotion estimation device including a learned emotion estimation model.

FIG. 2 is a diagram illustrating an overall configuration of an emotion estimation device according to a second embodiment of the present teaching.

FIG. 3 is a diagram illustrating a configuration of an emotion estimation model in the emotion estimation device according to the second embodiment of the present teaching.

FIG. 4 is a graph illustrating classification indexes of estimated emotion information in the emotion estimation device according to the present teaching.

FIG. 5 is a diagram illustrating a configuration when learning on a primary model used for learning of an emotion estimation system in the emotion estimation device according to the present teaching is performed.

FIG. 6 is a diagram illustrating a configuration after learning of the primary model used for learning of the emotion estimation system in the emotion estimation device according to the present teaching is finished.

FIG. 7 is a diagram illustrating a configuration when learning on a secondary-biological-information-distillation model included in the emotion estimation model in the emotion estimation device according to the second embodiment of the present teaching is performed using an output of the primary model.

FIG. 8 is a view illustrating a state where a user or a test subject wears the emotion estimation device of the present teaching.

FIG. 9 is a diagram illustrating a configuration when learning on a secondary-biological-information-distillation model included in an emotion estimation model in an emotion estimation device according to a third embodiment of the present teaching is performed using an output of a primary model.

FIG. 10 is a diagram illustrating a configuration when learning on a tertiary-biological-information-distillation model included in the emotional estimation model in the emotion estimation device of a fourth embodiment of the present teaching is performed using an output of the secondary-biological-information-distillation model.

### DESCRIPTION OF EMBODIMENTS

Embodiments will be described hereinafter with reference to the drawings. In the drawings, the same or corresponding parts are denoted by the same reference numerals, and description thereof will not be repeated. The dimensions of components in the drawings do not strictly represent actual dimensions of the components and dimensional proportions of the components, for example.

When an emotion is estimated from one type of biological information acquired from a user, a difference in the biological information due to a difference in the emotion of the user is unclear. Therefore, in a model that estimates an emotion based on only one type of biological information acquired from the user, in order to increase emotion estimation accuracy, a large amount of biological information for learning is needed, and moreover, even when the biological information for learning is increased, it is difficult to increase the emotion estimation accuracy. In other words, in general, in the technical field of emotion estimation based on biological information, in order to increase emotion estimation accuracy, a device or a method in which an emotion is estimated with an increased number of types of the biological information can be easily thought of. Reduction in the number of types of the biological information is, however, directly related to reduction in the emotion estimation accuracy of emotion estimation by the model, and therefore, a device or a method in which an emotion is estimated with a reduced number of types of the biological information is impractical from a viewpoint of common sense. Therefore, a device or a method in which emotion estimation accuracy is increased while the number of types of the biological information is reduced has yet to be proposed.

On the other hand, the plurality of types of the biological information acquired from the user are biological reaction occurring in a body of the user, and therefore, it is considered that each of the plurality of types of biological information is closely related to an emotion. For example, biological information, such as brain wave, sweating, respiration, electrocardiogram signal, or pulse wave of the user, each change as the emotion of the user changes. Therefore, in estimating the emotion of the user, it is considered that the model can complementarily use the plurality of types of the biological information each of which individually changes.

An nth-order-biological-information-distillation model in this specification can increase estimation accuracy even when a number of types of biological information of a user used for emotion estimation is reduced and thus convenience is increased. The nth-order-biological-information-distillation model is created using knowledge information obtained from a primary model or an (n−1)th-order model created complementarily using a plurality of types of biological information. This is considered to be because, in creating the nth-order-biological-information-distillation model that performs emotion estimation based on a small number of types of biological information of the user, the knowledge information functions to complement the small number of types of the biological information of the user. Thus, the inventors of the present teaching arrived at the present teaching that, centering on the biological information of the user, characteristics of the biological information closely related to an emotion of the user are utilized, so that emotion estimation accuracy is increased while the number of the types of the biological information is reduced to a smaller number by complementarily using the plurality of types of the biological information.

### First Embodiment

### <Emotion Estimation Based on Biological Information>

### <Overall Configuration of Emotion Estimation Device>

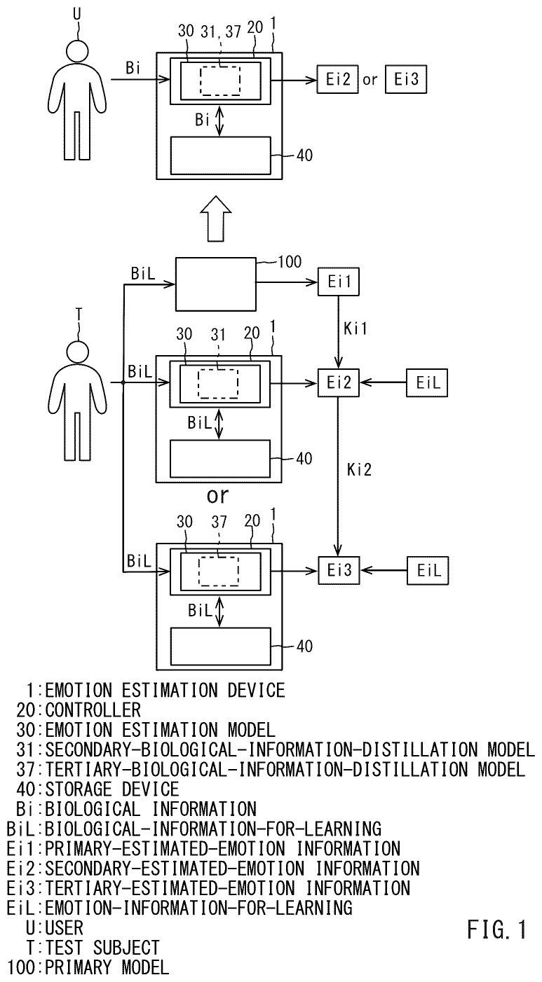

With reference to FIG. 1, an emotion estimation device 1 according to a first embodiment of the present teaching will be described. FIG. 1 is a diagram schematically illustrating learning on a secondary-biological-information-distillation model 31 or a tertiary-biological-information-distillation model 37 included in an emotion estimation model 30 in the emotion estimation device 1 according to the first embodiment of the present teaching and illustrates an overall configuration of the emotion estimation device 1 including the learned emotion estimation model 30.

As illustrated in FIG. 1, the emotion estimation device 1 according to the first embodiment of the present teaching estimates an emotion of a user U, based on biological information Bi of the user U (which will be hereinafter simply referred to as “biological information Bi”) or biological-information-for-learning BiL that is biological information of a test subject T (which will be hereinafter simply referred to as “biological-information-for-learning BiL). In this embodiment, the emotion estimation device 1 creates a secondary-estimated-emotion information Ei2 that is information related to the emotion of the user U from at least one type of the biological information Bi of the user U.

In each of the following embodiments, a model used for estimating the emotion based on a plurality of different types of the biological information Bi is defined as a primary model 100. The primary model 100 outputs primary knowledge information Ki1, based on the plurality of types of the biological information Bi. Models created using the primary knowledge information obtained based on the primary model 100 are defined as a secondary-biological-information-distillation model 31 and a secondary-biological-information-distillation model 34. Furthermore, a model created using secondary knowledge information Ki2 obtained based on the secondary-biological-information-distillation model 31 or the secondary-biological-information-distillation model 34 is defined as a tertiary-biological-information-distillation model 37. In this manner, a model created using (n−1)th-order-knowledge information obtained based on an (n−1)th-order-biological-information-distillation model is defined as an nth-order-biological-information-distillation model (n is each integer between 2 and m). n is a number expressing a generation of a biological-information-distillation model counted from the primary model assumed as a first generation.

The emotion estimation device 1 includes a controller 20 and a storage device 40. The controller 20 controls the emotion estimation device 1. The controller 20 creates a secondary-estimated-emotion information Ei2 or a tertiary-estimated-emotion information Ei3, based on the biological information Bi or the biological-information-for-learning BiL externally input thereto.

The controller 20 includes the emotion estimation model 30 used for estimating the emotion of the user U, based on the biological information Bi. The controller 20 is configured such that the biological information Bi or the biological-information-for-learning BiL can be externally input thereto.

In order to control the emotion estimation device 1 including the emotion estimation model 30 and the storage device 40, various types of programs and data are stored in the controller 20. The controller 20 is configured to be capable of transmitting the biological information Bi or the biological-information-for-learning BiL externally input thereto to the storage device 40. The controller 20 is configured to be capable of acquiring various types of information including the biological information Bi or the biological-information-for-learning BiL stored in the storage device 40 and information related to the emotion.

The emotion estimation model 30 is used for estimating the emotion of the user U, based on the biological information Bi. The emotion estimation model 30 is a model-for-machine-learning that has learned based on training data in which the biological-information-for-learning BiL and the emotion based on self-assessment of the test subject T are combined. The emotion estimation model 30 includes the secondary-biological-information-distillation model 31 created using the primary knowledge information Ki1 obtained based on the primary model 100 or the tertiary-biological-information-distillation model 37 created using the secondary knowledge information Ki2 obtained based on the secondary-biological-information-distillation model 31.

The secondary-biological-information-distillation model 31 is used for creating the secondary-estimated-emotion information Ei2 that is information expressing the emotion of the user U, based on the biological information Bi. The secondary-biological-information-distillation model 31 is configured such that the biological information Bi can be input thereto. The secondary-biological-information-distillation model 31 is configured to be capable of outputting the secondary-estimated-emotion information Ei2.

The tertiary-biological-information-distillation model 37 is used for creating the tertiary-estimated-emotion information Ei3 that is information expressing the emotion of the user U, based on the biological information Bi. The tertiary-biological-information-distillation model 37 is configured such that the biological information Bi can be input thereto. The tertiary-biological-information-distillation model 37 is configured to be capable of outputting the tertiary-estimated-emotion information Ei3.

The controller 20 is configured to be capable of outputting the secondary-estimated-emotion information Ei2 created using the secondary-biological-information-distillation model 31 of the emotion estimation model 30 or the tertiary-estimated-emotion information Ei3 created using the tertiary-biological-information-distillation model 37 of the emotion estimation model 30. The controller 20 is configured to be capable of outputting the secondary-estimated-emotion information Ei2 or the tertiary-estimated-emotion information Ei3 output by the emotion estimation model 30 to the storage device 40 and outside.

The storage device 40 stores the biological information Bi and the biological-information-for-learning BiL, as well as the secondary-estimated-emotion information Ei2 and the tertiary-estimated-emotion information Ei3 created by the controller 20 using the emotion estimation model 30, or the like. The storage device 40 includes a RAM that stores the biological information Bi, the biological-information-for-learning BiL, the secondary-estimated-emotion information Ei2, the tertiary-estimated-emotion information Ei3, or the like, a memory in a processor, a hard disk, or the like. The storage device 40 is configured to be capable of outputting the biological information Bi, the biological-information-for-learning BiL, the secondary-estimated-emotion information Ei2, and the tertiary-estimated-emotion information Ei3 stored therein to the controller 20.

In the emotion estimation device 1 configured in the above-described manner, the controller 20 causes the storage device 40 to store the biological information Bi externally input thereto. The controller 20 creates the secondary-estimated-emotion information Ei2 or the tertiary-estimated-emotion information Ei3 of the user U, based on the biological information Bi using the emotion estimation model 30. The controller 20 outputs the created secondary-estimated-emotion information Ei2 to the storage device 40 or the outside for every unit time.

### <Creation of Primary Model 100>

Next, the primary model 100 that is necessary for creating the emotion estimation model 30 will be described. The learned primary model 100 is a model that provides the primary knowledge information Ki1 created based on the plurality of different types of the biological information Bi to the emotion estimation model 30. In this embodiment, the primary model is a teacher model to the secondary-biological-information-distillation model 31.

The primary model 100 is a model-for-machine-learning used for estimating the emotion, based on a plurality of different types of the biological-information-for-learning BiL. The primary model 100 is a model that has learned based on training data including a combination of emotion-information-for-learning EiL that is information related to the emotion based on self-assessment of the test subject T and the biological-information-for-learning BiL including a plurality of different types of biological information. The emotion-information-for-learning EiL is an emotion of a correct answer in the training data.

The primary model 100 is used for creating primary-estimated-emotion information Ei1, based on the emotion-information-for-learning EiL. The primary model 100 is configured to be capable of outputting the primary-estimated-emotion information Ei1 when the emotion-information-for-learning EiL is input thereto. For the learned primary model 100, a parameter of the primary model is adjusted such that an error between the primary-estimated-emotion information Ei1 to the emotion-information-for-learning EiL input thereto and the emotion-information-for-learning EiL is as small as possible.

<Creation of Secondary-biological-information-distillation model 31>

Next, creation of the emotion estimation model 30 including the secondary-biological-information-distillation model 31 will be described. The emotion estimation model 30 is created by learning the primary knowledge information Ki1 obtained based on the biological-information-for-learning BiL including the plurality of different types of the biological information using the learned primary model 100, and the biological-information-for-learning BiL. In this embodiment, the emotion estimation model 30 uses as the primary knowledge information Ki1 the primary-estimated-emotion information Ei1 created based on the biological-information-for-learning BiL using the learned primary model 100. In this embodiment, the secondary-biological-information-distillation model 31 is a student model to the primary model 100.

The secondary-biological-information-distillation model 31 is configured to be capable of outputting the secondary-estimated-emotion information Ei2 when the emotion-information-for-learning EiL is input thereto. The secondary-biological-information-distillation model 31 is configured such that the parameter of the secondary-biological-information-distillation model 31 can be adjusted such that a total sum of an error of the secondary-estimated-emotion information Ei2 with respect to the emotion-information-for-learning EiL of the training data and an error of the secondary-estimated-emotion information Ei2 with respect to the primary-estimated-emotion information Ei1 that is the primary knowledge information Ki1 is as small as possible. That is, the secondary-biological-information-distillation model 31 performs learning using the primary-estimated-emotion information Ei1 obtained based on the biological-information-for-learning BiL using the learned primary model 100, the secondary-estimated-emotion information Ei2 obtained based on the biological-information-for-learning BiL using the secondary-biological-information-distillation model 31, and the emotion-information-for-learning EiL of the training data.

The secondary-biological-information-distillation model 31 is used for estimating the emotion of the user U, based on the biological-information-for-learning BiL that is included in types of the biological-information-for-learning BiL used in obtaining the primary knowledge information Ki1 (in estimating the emotion) using the primary model 100 and a number of the types of which is smaller than the number of the types of the biological-information-for-learning BiL necessary for estimating the emotion using the primary model 100.

The primary knowledge information Ki1 is an output of the primary model 100, obtained based on the plurality of types of the biological-information-for-learning BiL using the primary model 100. That is, the primary knowledge information Ki1 is information (knowledge of the primary model 100) used for estimating the emotion of the user U, obtained by the primary model 100 by learning based on the plurality of types of the biological-information-for-learning BiL.

By using the primary knowledge information Ki1, the secondary-biological-information-distillation model 31 performs learning in a state where the primary-estimated-emotion information Ei1 including information (privileged information) that is absent from the biological-information-for-learning BiL used for learning of the secondary-biological-information-distillation model 31 is inherited (distillation of knowledge).

Therefore, for the secondary-biological-information-distillation model 31 that performed learning using the primary knowledge information Ki1, in addition to the biological-information-for-learning BiL, emotion estimation accuracy can be increased to a higher level than that when learning was performed using only the biological-information-for-learning BiL. The secondary-biological-information-distillation model 31 can achieve estimation accuracy close to emotion estimation accuracy of the primary model 100 even when learning was performed based on the smaller number of types of the biological information than the number of types of the biological information used by the primary model 100 in obtaining the primary knowledge information Ki1 (a subset of types of the biological information).

Moreover, in the emotion estimation device 1, when learning of the secondary-biological-information-distillation model is performed using the primary knowledge information Ki1, a size of the model and a biological information amount necessary for estimating the emotion can be suppressed as compared to when learning of the secondary-biological-information-distillation model is performed without using the primary knowledge information. Accordingly, the emotion estimation device 1 can estimate the emotion of the user U with estimation accuracy close to emotion estimation accuracy of the primary model 100 by using the primary knowledge information Ki1 while suppressing increase in processing load of the hardware.

### <Creation of Tertiary-Biological-Information-Distillation Model>

Next, creation of the emotion estimation model 30 including the tertiary-biological-information-distillation model 37 will be described. The emotion estimation model 30 is created using the secondary knowledge information Ki2 obtained based on the plurality of types of the biological-information-for-learning BiL using the learned secondary-biological-information-distillation model 31, and the biological-information-for-learning BiL. In this embodiment, the emotion estimation model 30 uses, as the secondary knowledge information Ki2, the secondary-estimated-emotion information Ei2 created based on the biological-information-for-learning BiL using the learned secondary-biological-information-distillation model 31.

The tertiary-biological-information-distillation model 37 is configured to be capable of outputting the tertiary-estimated-emotion information Ei3 when the emotion-information-for-learning EiL is input thereto. The tertiary-biological-information-distillation model 37 is configured such that a parameter of the tertiary-biological-information-distillation model 37 can be adjusted such that a total sum of an error of the tertiary-estimated-emotion information Ei3 with respect to the emotion-information-for-learning EiL of the training data and an error of the tertiary-estimated-emotion information Ei3 with respect to the secondary-estimated-emotion information Ei2 that is the secondary knowledge information Ki2 is as small as possible. That is, the tertiary-biological-information-distillation model 37 performs learning using the secondary-estimated-emotion information Ei2 obtained based on the biological-information-for-learning BiL using the learned secondary-biological-information-distillation model 31, the tertiary-estimated-emotion information Ei3 obtained based on the biological-information-for-learning BiL using the tertiary-biological-information-distillation model 37, and the emotion-information-for-learning EiL of the training data.

The tertiary-biological-information-distillation model 37 is used for estimating the emotion of the user U, based on the biological-information-for-learning BiL that is included in types of the biological-information-for-learning BiL used in obtaining the secondary knowledge information Ki2 (in estimating the emotion) using the secondary-biological-information-distillation model 31 and a number of the types of which is smaller than the number of the types of the biological-information-for-learning BiL necessary for estimating the emotion using the secondary-biological-information-distillation model 31.

The controller 20 adjusts the parameter of the tertiary-biological-information-distillation model 37 such that the total sum of the error of the tertiary-estimated-emotion information Ei3 with respect to the emotion-information-for-learning EiL of the training data and the error of the tertiary-estimated-emotion information Ei3 with respect to the secondary-estimated-emotion information Ei2 that is the secondary knowledge information Ki2 of the secondary-biological-information-distillation model 31 is as small as possible.

The tertiary-biological-information-distillation model 37 configured in the above-described manner performs learning using the secondary-estimated-emotion information Ei2 obtained based on the biological-information-for-learning BiL that is the training data using the learned secondary-biological-information-distillation model 31, the tertiary-estimated-emotion information Ei3 obtained based on the biological-information-for-learning BiL using the tertiary-biological-information-distillation model 37, and the emotion-information-for-learning EiL of the training data.

The secondary knowledge information Ki2 is an output of the secondary-biological-information-distillation model 31 obtained based on at least one type of the biological-information-for-learning BiL using the secondary-biological-information-distillation model 31. That is, the secondary knowledge information Ki2 is information (knowledge of the tertiary-biological-information-distillation model 37) used for estimating the emotion of the user U, obtained by the secondary-biological-information-distillation model 31 by learning based on the plurality of different types of the biological-information-for-learning BiL.

By using the secondary knowledge information Ki2, the tertiary-biological-information-distillation model 37 performs learning in a state where the secondary-estimated-emotion information Ei2 including information (privileged information) that is absent from the biological-information-for-learning BiL used for learning of the tertiary-biological-information-distillation model 37 is inherited (distillation of knowledge).

Therefore, for the tertiary-biological-information-distillation model 37 that performed learning using the secondary knowledge information Ki2, in addition to the biological-information-for-learning BiL, emotion estimation accuracy can be increased to a higher level than that when learning was performed using only the biological-information-for-learning BiL. The tertiary-biological-information-distillation model 37 can achieve estimation accuracy close to emotion estimation accuracy of the secondary-biological-information-distillation model 31 even when learning was performed based on the smaller number of types of the biological information than the number of types of the biological information used by the secondary-biological-information-distillation model 31 in obtaining the secondary knowledge information Ki2 (a subset of types of the biological information).

The emotion estimation device 1 configured in the above-described manner can estimate the emotion of the user U with estimation accuracy close to the emotion estimation accuracy of the primary model 100 by using the primary knowledge information Ki1, while suppressing increase in processing load of the hardware. For example, the emotion estimation device 1 can easily acquire the biological information Bi of the user U preforming a specific work, action, or the like, such as the user U driving a four-wheel automobile and a motorcycle or operating a vessel, an airplane, various types of work machines, a drone, or the like, the walking user U, the user U during exercise such as running, or the user U watching a movie, appreciating a picture, viewing contents, or the like, by a wearable sensor or the like, and estimate the emotion of the user U preforming the specific work, action, or the like. The biological information that can be easily acquired refers to, for example, biological information measurable by a user alone, biological information acquirable using a single or easy-to-operate sensor, or biological information measurable without being affected by a surrounding environment or the user's condition. The biological information that is difficult to acquire easily refers to, for example, biological information requiring measurement by both the user and an operator, biological information acquired using a plurality of sensors or a sensor with complex operation, or biological information that is likely to be affected by the surrounding environment or the user's condition.

### Second Embodiment

### <Emotion Estimation Based on Electrocardiogram Signal and Pulse Wave>

### <Overall Configuration of Emotion Estimation Device>

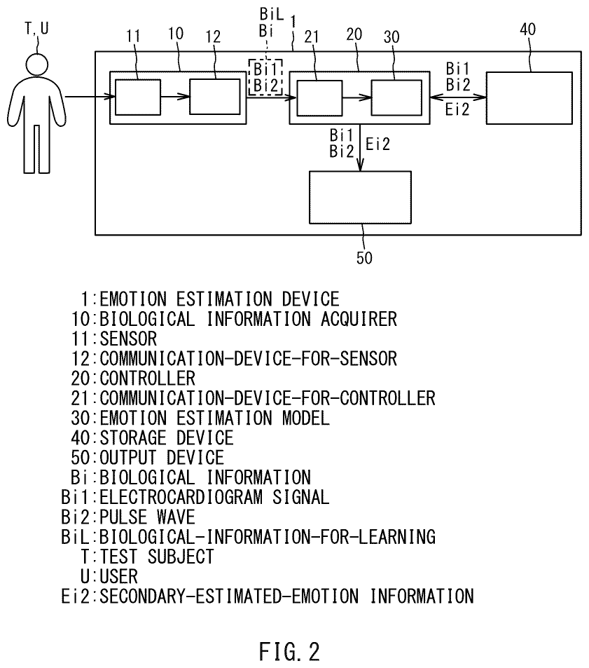

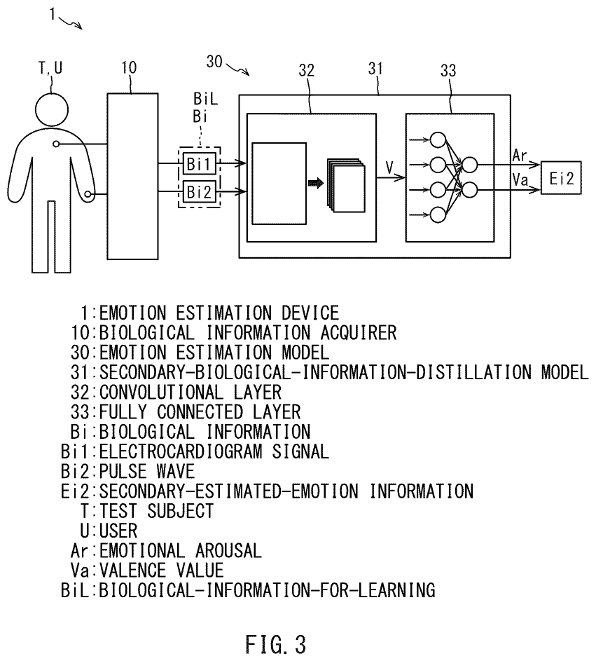

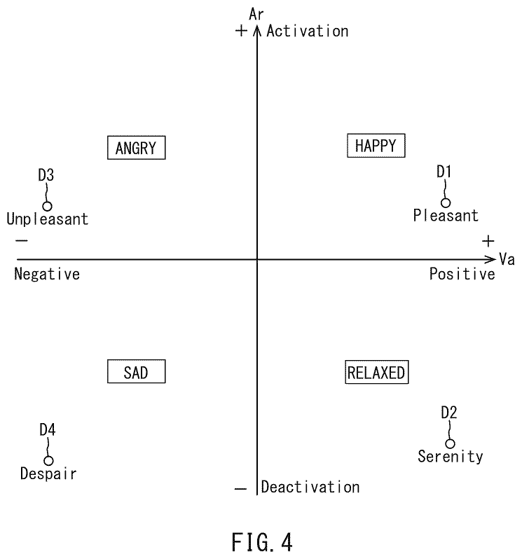

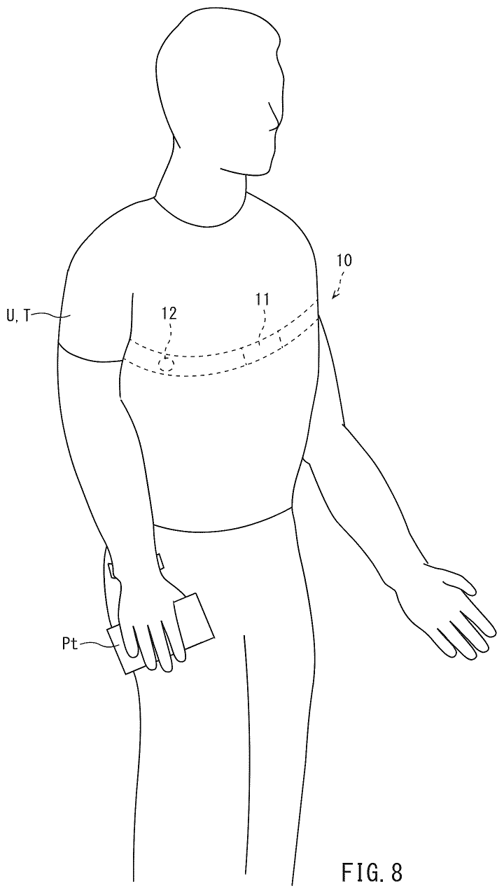

With reference to FIG. 2 to FIG. 4 and FIG. 8, an emotion estimation device 1 according to a second embodiment of the emotion estimation device of the present teaching will be described. FIG. 2 illustrates an overall configuration of the emotion estimation device 1 according to the second embodiment of the present teaching. FIG. 3 illustrates a configuration of an emotion estimation model 30 in the emotion estimation device 1. FIG. 4 is a graph illustrating classification indexes of a secondary-estimated-emotion information Ei2 in the emotion estimation device 1. FIG. 8 illustrates a state where a user U or a test subject T wears the emotion estimation device 1. The emotion estimation device 1 according to the second embodiment is an emotion estimation device that discloses a configuration of the emotion estimation device 1 of the first embodiment in more detail. Note that, in the following embodiment, specific description of similar points to those in the embodiment already described will be omitted and only a portion which differs from the already described embodiment will be described in detail.

As illustrated in FIG. 2, the emotion estimation device 1 according to the second embodiment of the present teaching estimates an emotion of the user U, based on biological information Bi of the user U illustrated in FIG. 8. In this embodiment, the emotion estimation device 1 creates estimated emotion information that is information related to the emotion of the user U from the biological information Bi related to electrocardiogram signal and pulse wave of the user U.

The emotion estimation device 1 includes a biological information acquirer 10, a controller 20, a storage device 40, and an output device 50. The storage device 40, the output device 50, and the controller 20 of the emotion estimation device 1 are built in a portable terminal Pt (see FIG. 8), such as a smartphone, that is carried by the user U.

That is, the emotion estimation device 1 shares a portion of a configuration of the portable terminal Pt. Specifically, a memory of the portable terminal Pt functions as the storage device 40 of the emotion estimation device 1. A liquid crystal monitor of the portable terminal Pt functions as the output device 50 of the emotion estimation device 1. A processor of the portable terminal Pt functions as the controller 20 of the emotion estimation device 1.

The biological information acquirer 10 acquires (measures) biological information Bi or biological-information-for-learning BiL. The biological information acquirer 10 outputs the acquired biological information Bi or biological-information-for-learning BiL to the controller 20. The biological information acquirer 10 includes a sensor 11, such as an unillustrated heart rate sensor, that acquires the biological information Bi or the biological-information-for-learning BiL, and a communication-device-for-sensor 12 that transmits and receives a control signal, the acquired biological information Bi or biological-information-for-learning BiL, or the like.

The biological information acquirer 10 acquires, for example, at least one of electrocardiogram signal, heart rate, heart sound, heart rate waveform, cardiac cycle, heart rate change, blood pressure, pulse wave, triaxial acceleration, body surface temperature, brain wave, respiratory rate, pupil state, myoelectric potential, blood component, exhalation, exhalation amount, exhalation component, or the like that is the biological information Bi or the biological-information-for-learning BiL using the sensor 11.

In this embodiment, the biological information acquirer 10 acquires biological information Bi1 related to the electrocardiogram signal (which will be hereinafter simply referred to as “electrocardiogram signal Bi1”) and biological information Bi2 related to the pulse wave (which will be hereinafter simply referred to as “pulse wave Bi2”), which are biological information related to the heat beat, among the biological information Bi or the biological-information-for-learning BiL. The biological information acquirer 10 acquires the electrocardiogram signal Bi1 and the pulse wave Bi2 of the user U or the test subject T for every unit time, for example, by a wearable sensor that is the sensor 11 that can be easily worn by the user U or the test subject T. Thus, the biological information acquirer 10 outputs the biological information Bi including the electrocardiogram signal Bi1 and the pulse wave Bi2 of the user U or the test subject T acquired by the sensor 11 to the controller 20 using the communication-device-for-sensor 12.

The controller 20 controls the biological information acquirer 10, the storage device 40, and the output device 50. The controller 20 also creates estimated emotion information, based on the biological information Bi of the user U input from the biological information acquirer 10.

The controller 20 includes the emotion estimation model 30 used for estimating the emotion of the user U, based on the electrocardiogram signal Bi1 and the pulse wave Bi2 of the user U or the test subject T. The controller 20 includes a communication-device-for-controller 21 that transmits and receives a control signal or the like to and from the biological information acquirer 10 and acquires the electrocardiogram signal Bi1 and the pulse wave Bi2 of the user U or the test subject T.

In order to control the biological information acquirer 10, the storage device 40, the output device 50, and the emotion estimation model 30, various types of programs and data are stored in the controller 20. The controller 20 is configured to be capable of transmitting and receiving the control signal to and from the biological information acquirer 10 via the communication-device-for-controller 21. Similarly, the controller 20 is configured to be capable of transmitting and receiving the control signal to and from the storage device 40 and the output device 50.

The controller 20 is configured to be capable of transmitting the biological information Bi or the biological-information-for-learning BiL acquired by the biological information acquirer 10 to the storage device 40. The controller 20 is configured to be capable of acquiring various types of information including the biological information Bi of the user U stored in the storage device 40 and information related to the emotion. The controller 20 is configured to be cable of outputting the various types of information including the biological information Bi of the user U and the information related to the emotion to the output device 50.

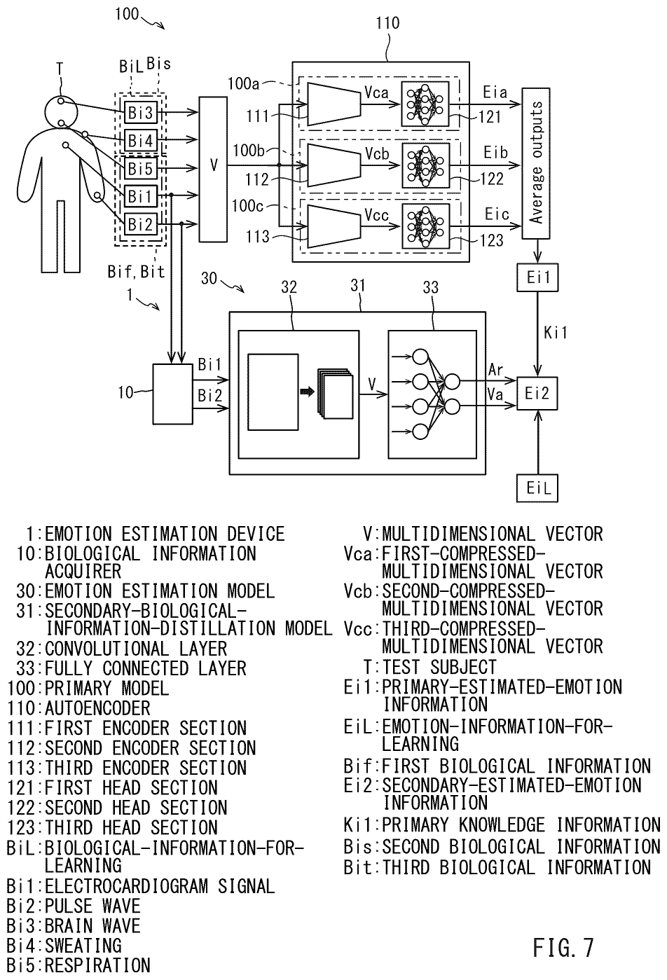

The emotion estimation model 30 estimates the emotion of the user U, based on the electrocardiogram signal Bi1 and the pulse wave Bi2 of the user U. The emotion estimation model 30 is a neural network model that has learned based on training data in which the electrocardiogram signal Bi1 and the pulse wave Bi2 (see FIG. 3) that are the biological-information-for-learning BiL and the emotion based on self-assessment of the test subject T are combined. The emotion estimation model 30 includes a secondary-biological-information-distillation model 31 that has learned primary knowledge information Ki1 (see FIG. 7) created using a primary model 100 (see FIG. 3).

As illustrated in FIG. 3, the emotion estimation model 30 outputs the emotion of the user U estimated based on the electrocardiogram signal Bi1 and the pulse wave Bi2 of the user U using the secondary-biological-information-distillation model 31, as secondary-estimated-emotion information Ei2 represented by emotional arousal Ar (Arousal) representing a strength of the emotion and valence value Va (Valence) representing a degree of emotional positivity.

The secondary-biological-information-distillation model 31 included in the emotion estimation model 30 is a convolutional neural network model (CNN) that extracts features of image data of the electrocardiogram signal Bi1 and the pulse wave Bi2 and classifies the image data, based on the features. The secondary-biological-information-distillation model 31 includes a convolutional layer 32, a fully connected layer 33, or the like.

The convolutional layer 32 is used for extracting the features of the image data of the electrocardiogram signal Bi1 and the pulse wave Bi2. The convolutional layer 32 is configured such that the electrocardiogram signal Bi1 and the pulse wave Bi2 of the user U acquired by the sensor 11 can be input thereto.

The secondary-biological-information-distillation model 31 is configured to be capable of calculating, when the electrocardiogram signal Bi1 and the pulse wave Bi2 of the user U is input to the convolutional layer 32, a multidimensional vector V that is a group of a plurality of features of each of the electrocardiogram signal Bi1 and the pulse wave Bi2 of the user U, including time-domain features, such as an R-R interval, a mean R-R interval, or a standard deviation of R-R intervals; frequency-domain features, such as a low-frequency range power spectrum (LF), a high-frequency range power spectrum (HF), or an LF/HF ratio; or nonlinear-domain features, such as sample entropy or a Lyapunov exponent, by using the convolutional layer 32. The convolutional layer 32 outputs the calculated multidimensional vectors V of the electrocardiogram signal Bi1 and the pulse wave Bi2 to the fully connected layer 33.

The fully connected layer 33 is used for estimating the emotion of the user U, based on the multidimensional vector V. The fully connected layer 33 is used for creating the secondary-estimated-emotion information Ei2 that is information related to the emotion, based on the input multidimensional vector V.

The secondary-estimated-emotion information Ei2 includes the emotional arousal Ar and the valence value Va of the estimated emotion. The controller 20 is configured to be capable of outputting the secondary-estimated-emotion information Ei2 created using the secondary-biological-information-distillation model 31. The controller 20 is configured to be capable of outputting the secondary-estimated-emotion information Ei2 output by the emotion estimation model 30 to the storage device 40 and the output device 50 (see FIG. 2).

As illustrated in FIG. 2, the storage device 40 stores the biological information Bi acquired by the sensor 11 included in the biological information acquirer 10, the secondary-estimated-emotion information Ei2 created by the controller 20 using the emotion estimation model 30, or the like. The storage device 40 includes a RAM that stores the biological information Bi and the secondary-estimated-emotion information Ei2, a memory in a processor, a hard disk, or the like.

In this embodiment, the storage device 40 is a memory of the portable terminal Pt. The storage device 40 is configured to be capable of acquiring a control signal or the like from the controller 20. The storage device 40 is configured to be capable of storing the biological information Bi input from the biological information acquirer 10 via the controller 20, as well as the secondary-estimated-emotion information Ei2 and information related to the user U input from the controller 20, in time series. The storage device 40 is configured to be capable of outputting the stored biological information Bi and secondary-estimated-emotion information Ei2 to the controller 20.

The output device 50 outputs at least one of the biological information Bi or the secondary-estimated-emotion information Ei2 to the user U. The output device 50 includes a display device, a speaker, or the like. In this embodiment, the output device 50 is a liquid crystal monitor of the portable terminal Pt (see FIG. 8).

The output device 50 is configured to be capable of acquiring the control signal or the like from the controller 20. The output device 50 is configured to be capable of acquiring the biological information Bi and the secondary-estimated-emotion information Ei2 from the controller 20. The output device 50 is configured to be capable of outputting the acquired biological information Bi and secondary-estimated-emotion information Ei2 to the liquid crystal monitor or the like of the portable terminal Pt via at least one of graphic information, text information, or color information.

The emotion estimation device 1 configured in the above-described manner includes the biological information acquirer 10 including the sensor 11, the controller 20 configured of the portable terminal Pt of the user U, the storage device 40, and the output device 50. The controller 20 of the emotion estimation device 1 acquires the biological information Bi by the biological information acquirer 10 for every unit time. The controller 20 causes the storage device 40 to store the acquired biological information Bi. The controller 20 creates the secondary-estimated-emotion information Ei2 of the user U for every unit time, based on the acquired biological information Bi, using the emotion estimation model 30. At this time, the controller 20 may use information related to the user U acquired in advance for estimating the emotion. The controller 20 outputs the created secondary-estimated-emotion information Ei2 to the storage device 40 and the output device 50 for every unit time.

As illustrated in FIG. 4, the controller 20 displays the emotion of the user U to the output device 50, based on the secondary-estimated-emotion information Ei2, using an emotion circumplex (see FIG. 3). In the emotion circumplex, emotions are classified based on the emotional arousal Ar and the valence value Va of each of the emotions. The controller 20 converts the emotion of the user U classified based on the emotional arousal Ar and the valence value Va of the emotion using the emotion circumplex to at least one of graphic information, text information, or color information, and outputs the converted emotion to the output device 50.

The valence value Va represents a degree of emotional positivity between a positive emotion and a negative emotion. Positive emotions refer to affirmative mental states, such as excitement, relief, elation, serenity, calmness, or pleasantness. Negative emotions refer to negative mental states, such as tension, agitation, melancholy, anxiety, unpleasantness, or despair.

When the valence value Va is positive, the controller 20 determines that the user U has a positive emotion in accordance with a degree of the valence value Va. When the valence value Va is negative, the controller 20 determines that the user U has a negative emotion in accordance with the degree of the valence value Va.

The emotional arousal Ar represents a degree of the emotion between a state where the emotion is activated and a state where the emotion is deactivated. Activated emotions refer to active mental states, such as excitement, elation, pleasantness, agitation, or tension. Deactivated emotions refer to passive mental states, such as relief, serenity, calmness, melancholy, or boredom.

When the emotional arousal Ar is positive, the controller 20 determines that the user U has an activated emotion in accordance with a degree of the emotional arousal Ar. When the emotional arousal Ar is negative, the controller 20 determines that the user U has a deactivated emotion in accordance with the degree of the emotional arousal Ar.

When the valence value Va is positive and the emotional arousal Ar is positive, the controller 20 allocates the emotion of the user U to an emotion included in a happy area (Happy) including emotions of excitement, elation, pleasantness, or the like. For example, when the valence value Va is high and the emotional arousal Ar is low in the happy area, the controller 20 selects an emotion of pleasantness (Pleasant) from a plurality of types of emotions in the happy aera.

When the valence value Va is positive and the emotional arousal Ar is negative, the controller 20 allocates the emotion of the user U to an emotion included in a relaxed area (Relaxed) including relief, serenity, calmness, or the like. For example, when the valence value Va is high and the emotional arousal Ar is low in the relaxed area, the controller 20 selects an emotion of serenity (Serenity) from a plurality of types of emotions in the relaxed area.

When the valence value Va is negative and the emotional arousal Ar is positive, the controller 20 allocates the emotion of the user U to an emotion included in an angry area (Angry) including tension, agitation, unpleasantness, or the like. For example, when the valence value Va is low and the emotional arousal Ar is low in the angry area, the controller 20 selects an emotion of unpleasantness (Unpleasant) from a plurality of types of emotions in the angry area.

When the valence value Va is negative and the emotional arousal Ar is negative, the controller 20 allocates the emotion of the user U to an emotion included in a sad area (Sad) including melancholy, anxiety, despair, or the like. For example, when the valence value Va is low and the emotional arousal Ar is low in the sad area, the controller 20 selects an emotion of despair (Despair) from a plurality of types of emotions in the sad area.

### <Creation of Primary Model>

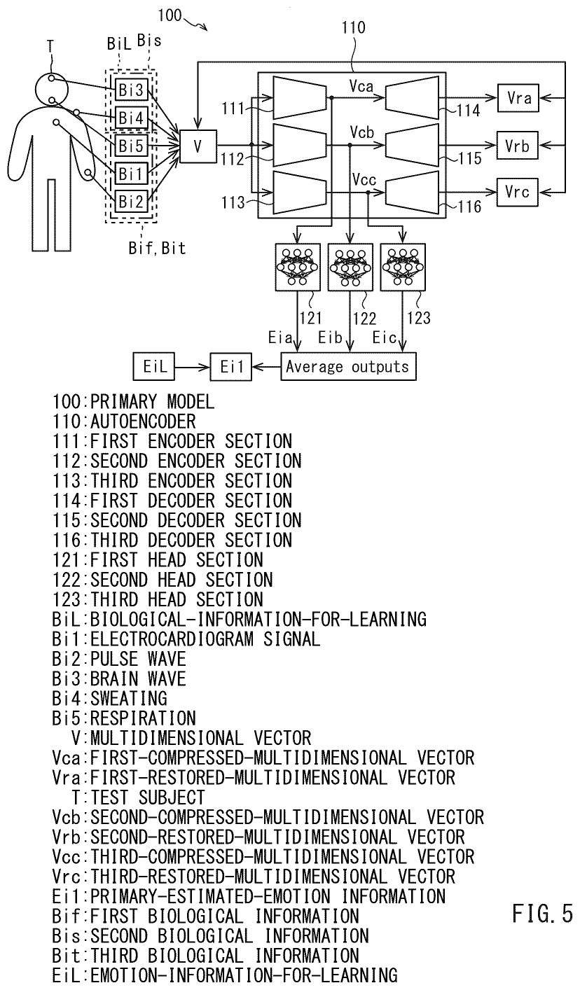

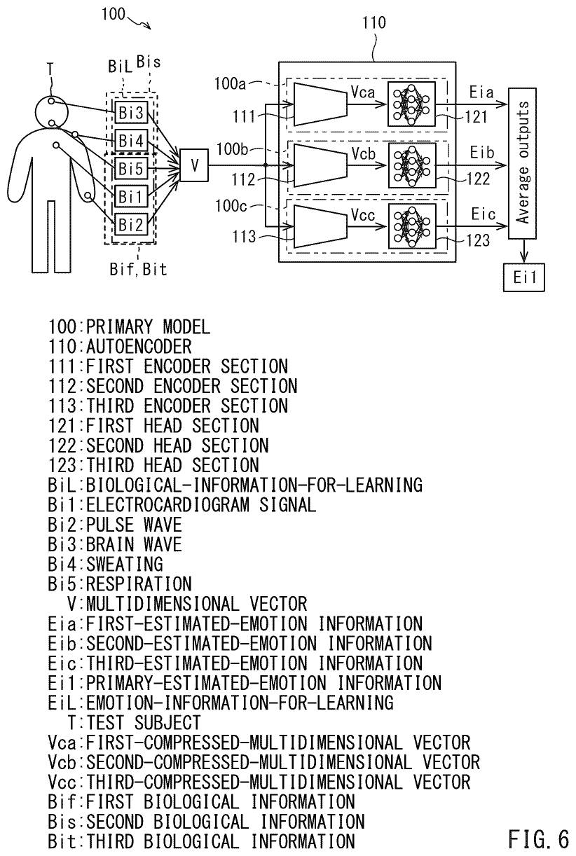

Next, with reference to FIG. 5 and FIG. 6, the primary model 100 necessary for creating the emotion estimation model 30 will be described. FIG. 5 illustrates a configuration when learning of the primary model 100 used for learning of the emotion estimation model 30 (see FIG. 2) in the emotion estimation device 1 is performed. FIG. 6 illustrates a configuration after learning of the primary model 100 used for learning of the emotion estimation model 30 in the emotion estimation device 1 is finished. The learned primary model 100 is a teacher model that provides the primary knowledge information Ki1 created based on a plurality of different types of the biological information Bi to the emotion estimation model 30.

As illustrated in FIG. 5, the primary model 100 is a neural network model used for estimating an emotion, based on a plurality of different types of the biological-information-for-learning BiL. The primary model 100 is a model that has learned based on training data including a combination of the emotion-information-for-learning EiL that is information related to the emotion based on self-assessment of the test subject T and the plurality of different types of the biological-information-for-learning BiL of the test subject T, which are the electrocardiogram signal Bi1, the pulse wave Bi2, biological information Bi3 related to the brain wave (which will be hereinafter simply referred to as a “brain wave Bi3”), biological information Bi4 related to sweating (which will be hereinafter simply referred to as “sweating Bi4”), and biological information Bi5 related to respiration (which will be hereinafter simply referred to as “respiration Bi5”). The emotion-information-for-learning EiL is an emotion of a correct answer in the training data. The emotion-information-for-learning EiL includes the emotional arousal Ar and the valence value Va of the emotion.

In this embodiment, at least one of the electrocardiogram signal Bi1, the pulse wave Bi2, or the respiration Bi5, which is the biological information Bi that is less likely to be affected by a surrounding environment of the user U, a work of the user U, an action of the user U, or the like, the biological information Bi that can be easily acquired from the user U by the wearable sensor or the like, or the biological information Bi that is less likely to be affected by the surrounding environment of the user U, the work of the user U, the action of the user U, or the like and can be easily acquired from the user U by the wearable sensor or the like, is defined as first biological information Bif.

In this embodiment, at least one of the brain wave Bi3 or the sweating Bi4, which is the biological information Bi that is likely to be affected by the surrounding environment of the user U, the work of the user U, or the action of the user U, the biological information Bi that is difficult to acquire easily from the user U, or the biological information Bi that is likely to be affected by the surrounding environment of the user U, the work of the user U or the action of the user U and is difficult to acquire easily from the user U, is defined as second biological information Bis.

In this embodiment, third biological information Bit includes, for example, at least two of the brain wave Bi3, the sweating Bi4, the respiration Bi5, the pulse wave Bi2, or the electrocardiogram signal Bi1 that are biological information that seems to be related to the emotion of the user. For example, the electrocardiogram signal Bi1, the pulse wave Bi2, and the respiration Bi5, which are a combination of three of the brain wave Bi3, the sweating Bi4, the respiration Bi5, the pulse wave Bi2, or the electrocardiogram signal Bi1 that are biological information that seem to be related to the emotion of the user, are defined as the third biological information Bit. In this embodiment, the first biological information Bif and the third biological information Bit includes the same type of the biological information Bi.

Learning is performed on the primary model 100, based on the multidimensional vector V of features of each of the electrocardiogram signal Bi1, the pulse wave Bi2, and the respiration Bi5 that are the first biological information Bif, and the brain wave Bi3 and the sweating Bi4 that are the second biological information Bis, as well as the emotion-information-for-learning EiL. That is, learning is performed on the primary model 100, based on the multidimensional vector V of the features of each of the electrocardiogram signal Bi1, the pulse wave Bi2, the respiration Bi5, the brain wave Bi3 and the sweating Bi4, which constitute the plurality of types of the biological-information-for-learning BiL including the electrocardiogram signal Bi1, the pulse wave Bi2 and the respiration Bi5 that are defined as the third biological information Bit, as well as the emotion-information-for-learning EiL.

The features of the electrocardiogram signal Bi1 and the pulse wave Bi2 include mean and standard deviation of R-R intervals, low-frequency range and high-frequency range power spectra, a power spectral ratio, sample entropy, a Lyapunov exponent, or the like. The features of the brain wave Bi3 includes root mean squares, Hjorth parameters representing mobility and complexity, power spectra, mean and standard deviations of phase synchronization indexes, or the like, in θ (theta) waves, α (alpha) waves, β (beta) waves, and γ (gamma) waves. The features of the sweating Bi4 includes an amplitude and a peak time in the skin conductance response; a tonic level in the skin electrical response; mean, standard deviation, root mean square, and power spectrum of the phasic response; mean and standard deviation of the Mel-frequency cepstral coefficient, or the like. The features of the respiration Bi5 includes mean, standard deviation, maximum, and minimum values at the zero-crossing time, low-frequency range and high-frequency range power spectra, a power spectral ratio, sample entropy, a Lyapunov exponent, or the like.

The primary model 100 includes an autoencoder 110 that is a neural network model, and a first head section 121, a second head section 122, and a third head section 123 that are fully connected layers with the neural network model.

The autoencoder 110 is a model that extracts features of the multidimensional vector V by reducing dimensions (a number of variables) of the multidimensional vector V including a plurality of variables and then restoring the dimensions of the multidimensional vector V to original dimensions. The autoencoder 110 includes a plurality of encoder sections and a plurality of decoder sections. In this embodiment, the autoencoder 110 includes a combination of a first encoder section 111 and a first decoder section 114, a combination of a second encoder section 112 and a second decoder section 115, and a combination of a third encoder section 113 and a third decoder section 116 in order to perform ensemble learning. The primary model 100 is configured to be capable of inputting the multidimensional vector V created based on the biological-information-for-learning BiL to each of the first encoder section 111, the second encoder section 112, and the third encoder section 113 of the autoencoder 110.

The first encoder section 111, the second encoder section 112, and the third encoder section 113 are dimensionality reducers that each reduce the dimensions of the multidimensional vector V including the plurality of variables using a function. That is, the first encoder section 111, the second encoder section 112, and the third encoder section 113 compress the multidimensional vector V to a lower dimensional vector using a function. As described above, the first encoder section 111, the second encoder section 112, and the third encoder section 113 are each configured to be usable for creating a first-compressed-multidimensional vector Vca, a second-compressed-multidimensional vector Vcb, and a third-compressed-multidimensional vector Vcc obtained by extracting an important feature from the multidimensional vector V.

The first encoder section 111 is configured to be capable of outputting the first-compressed-multidimensional vector Vca to the first decoder section 114 and the first head section 121. The second encoder section 112 is configured to be capable of outputting the second-compressed-multidimensional vector Vcb to the second decoder section 115 and the second head section 122. The third encoder section 113 is configured to be capable of outputting the third-compressed-multidimensional vector Vcc to the third decoder section 116 and the third head section 123.

The first decoder section 114, the second decoder section 115, and the third decoder section 116 are dimensionality decoders that restore dimensions of the first-compressed-multidimensional vector Vca, the second-compressed-multidimensional vector Vcb, and the third-compressed-multidimensional vector Vcc, dimensions of which are reduced using the function and each of which includes a plurality of variables, to dimensions before dimension reduction.

The first decoder section 114 is configured to be usable for creating a first-restored-multidimensional vector Vra that is obtained by restoring the dimensions of the first-compressed-multidimensional vector Vca compressed by the first encoder section 111 to the dimensions before compression. The second decoder section 115 is configured to be usable for creating a second-restored-multidimensional vector Vrb that is obtained by restoring the dimensions of the second-compressed-multidimensional vector Vcb compressed by the second encoder section 112 to the dimensions before compression. The third decoder section 116 is configured to be usable for creating a third-restored-multidimensional vector Vrc that is obtained by restoring the dimensions of the third-compressed-multidimensional vector Vcc compressed by the third encoder section 113 to the dimensions before compression.

Each of the first head section 121, the second head section 122, and the third head section 123 is a fully connected layer used for estimating the emotion of the test subject T, based on all of the variables that are input to a corresponding one of the first head section 121, the second head section 122, and the third head section 123. The first head section 121 is used for estimating the emotion of the test subject T, based on all of the first-compressed-multidimensional vectors Vca input from the first encoder section 111. That is, the first head section 121 is configured to be usable for creating first-estimated-emotion information Eia including the emotional arousal Ar and the valence value Va of the emotion, based on the first-compressed-multidimensional vector Vca.

Similarly, the second head section 122 is configured to be usable for creating second-estimated-emotion information Eib, based on the second-compressed-multidimensional vector Vcb. The third head section 123 is configured to be usable for creating third-estimated-emotion information Eic, based on the third-compressed-multidimensional vector Vcc.

Furthermore, the primary model 100 is configured to be usable for creating primary-estimated-emotion information Ei1, based on the first-estimated-emotion information Eia created by the first head section 121, the second-estimated-emotion information Eib created by the second head section 122, and the third-estimated-emotion information Eic created by the third head section 123. The primary-estimated-emotion information Ei1 includes the emotional arousal Ar and the valence value Va of the emotion. In this embodiment, the primary-estimated-emotion information Ei1 is an average of the first-estimated-emotion information Eia, the second-estimated-emotion information Eib, and the third-estimated-emotion information Eic.

In the primary model 100, parameters of the functions in the first encoder section 111 and the first decoder section 114 are adjusted such that an error between the multidimensional vector V that is input to the first encoder section 111 and the first-restored-multidimensional vector Vra created using the first decoder section 114 is further reduced. In the primary model 100, parameters of the functions in the second encoder section 112 and the second decoder section 115 are adjusted such that an error between the multidimensional vector V that is input to the second encoder section 112 and the second-restored-multidimensional vector Vrb created using the second decoder section 115 is further reduced. In the primary model 100, parameters of the functions in the third encoder section 113 and the third decoder section 116 are adjusted such that an error between the multidimensional vector V that is input to the third encoder section 113 and the third-restored-multidimensional vector Vrc created using the third decoder section 116 is further reduced.

In the primary model 100, parameters of the functions in the first head section 121, the second head section 122, and the third head section 123 are adjusted such that an error between the emotion-information-for-learning EiL included in the training data and the primary-estimated-emotion information Ei1 is further reduced.

The primary model 100 configured in the above-described manner performs learning using, as training data, a plurality of combinations of the respective multidimensional vectors V of the electrocardiogram signal Bi1, the pulse wave Bi2, the brain wave Bi3, the sweating Bi4 and the respiration Bi5, and the emotion-information-for-learning EiL.

In the primary model 100, the multidimensional vectors V of the plurality of different types of the biological-information-for-learning BiL of the test subject T are input to the autoencoder 110. In the primary model 100, the first-restored-multidimensional vector Vra, the second-restored-multidimensional vector Vrb, and the third-restored-multidimensional vector Vrc with respect to the multidimensional vector V are created using the autoencoder 110. Along with that, in the primary model 100, the primary-estimated-emotion information Ei1 is created using the first encoder section 111 and the first head section 121, the second encoder section 112 and the second head section 122, and the third encoder section 113 and the third head section 123.

In the primary model 100, parameters of the autoencoder 110 and the parameters of the first head section 121, the second head section 122, and the third head section 123 are adjusted (learning is performed) such that a total sum of an error of the first-restored-multidimensional vector Vra with respect to the multidimensional vector V, an error of the second-restored-multidimensional vector Vrb with respect to the multidimensional vector V, an error of the third-restored-multidimensional vector Vrc with respect to the multidimensional vector V, and an error of the primary-estimated-emotion information Ei1 with respect to the emotion-information-for-learning EiL is as small as possible.

As illustrated in FIG. 6, in the primary model 100, when learning is finished, the first decoder section 114, the second decoder section 115, and the third decoder section 116 are removed. Therefore, the primary model 100 includes a first internal model 100a obtained by combining the first encoder section 111 and the first head section 121, a second internal model 100b obtained by combining the second encoder section 112 and the second head section 122, and a third internal model 100c obtained by combining the third encoder section 113 and the third head section 123.

Thus, the primary model 100 is configured such that each of a plurality of internal models, that is, the first internal model 100a, the second internal model 100b, and the third internal model 100c, is usable for estimating the emotion of the test subject T, based on the electrocardiogram signal Bi1, the pulse wave Bi2, the brain wave Bi3, the sweating Bi4, and the respiration Bi5 that are the biological-information-for-learning BiL. Moreover, the primary model 100 is configured to be capable of outputting the primary-estimated-emotion information Ei1 that is an average of the first-estimated-emotion information Eia created using the first internal model 100a, the second-estimated-emotion information Eib created using the second internal model 100b, and the third-estimated-emotion information Eic created using the third internal model 100c.

### <Creation of Secondary-Biological-Information-Distillation Model>

Next, with reference to FIG. 7, creation of the emotion estimation model 30 will be described. FIG. 7 illustrates a configuration when learning on the secondary-biological-information-distillation model 31 included in the emotion estimation model 30 in the emotion estimation device 1 is performed using an output of the primary model 100.

As illustrated in FIG. 7, the emotion estimation model 30 is created by learning the primary knowledge information Ki1 obtained based on the multidimensional vectors V of the biological-information-for-learning BiL including a plurality different types of biological information using the learned primary model 100, and the biological-information-for-learning BiL. In this embodiment, the emotion estimation model 30 uses, as the primary knowledge information Ki1, the primary-estimated-emotion information Ei1 created based on the multidimensional vectors V of the biological-information-for-learning BiL using the learned primary model 100.

The emotion estimation model 30 includes the secondary-biological-information-distillation model 31 created by learning the primary knowledge information Ki1 obtained based on the multidimensional vectors V of the biological-information-for-learning BiL including a plurality of different types of biological information using the learned primary model 100. The secondary-biological-information-distillation model 31 includes the convolutional layer 32 used for calculating the multidimensional vectors V of the biological information Bi and the biological-information-for-learning BiL and the fully connected layer 33 used for calculating the secondary-estimated-emotion information Ei2 including the emotional arousal Ar and the valence value Va of the emotion estimated based on the multidimensional vector V.

The secondary-biological-information-distillation model 31 is configured such that parameters of the convolutional layer 32 and the fully connected layer 33 can be adjusted such that an overall error L obtained by summing an error L1 of the secondary-estimated-emotion information Ei2 with respect to the emotion-information-for-learning EiL of the training data and an error L2 of the secondary-estimated-emotion information Ei2 with respect to the primary-estimated-emotion information Ei1 that is the primary knowledge information Ki1 of the primary model 100 at an arbitrary rate a is as small as possible (see Expression 1).

$$L = α \cdot L 1 + ( 1 - α ) \cdot L 2 \qquad [ Expression 1 ]$$

In the secondary-biological-information-distillation model 31 configured in the above-described manner, learning is performed using the primary-estimated-emotion information Ei1 that is the primary knowledge information Ki1 obtained based on the multidimensional vectors V of the biological-information-for-learning BiL that is the training data using the learned primary model 100, the secondary-estimated-emotion information Ei2 obtained based on the biological-information-for-learning BiL using the secondary-biological-information-distillation model 31, and the emotion-information-for-learning EiL of the training data.

In this embodiment, in the primary model 100, learning is performed using, in addition to the electrocardiogram signal Bi1, the pulse wave Bi2, and the respiration Bi5 that are the first biological information Bif in the biological-information-for-learning BiL of the plurality of test subject T, the brain wave Bi3 and the sweating Bi4 that are the second biological information Bis as the training data. In performing learning on the secondary-biological-information-distillation model 31, the electrocardiogram signal Bi1, the pulse wave Bi2, the brain wave Bi3, the sweating Bi4, and the respiration Bi5 are used for the primary model 100. In the secondary-biological-information-distillation model 31, the training data, which includes the electrocardiogram signal Bi1 and the pulse wave Bi2 included in the first biological information Bif in the biological-information-for-learning BiL and the emotion-information-for-learning EiL that is information related to the emotion based on self-assessment of the test subject T, is used.

In performing learning on the secondary-biological-information-distillation model 31, in the primary model 100, the multidimensional vectors V of the electrocardiogram signal Bi1, the pulse wave Bi2, the brain wave Bi3, the sweating Bi4, and the respiration Bi5 that are the biological-information-for-learning BiL are input to each of the first internal model 100a including the first encoder section 111 and the first head section 121, the second internal model 100b including the second encoder section 112 and the second head section 122, and the third internal model 100c including the third encoder section 113 and the third head section 123. In the primary model 100, the first-estimated-emotion information Eia is created using the first encoder section 111 and the first head section 121, the second-estimated-emotion information Eib is created using the second encoder section 112 and the second head section 122, and the third-estimated-emotion information Eic is created using the third encoder section 113 and the third head section 123.

In the primary model 100, the primary-estimated-emotion information Ei1 that is an average of the first-estimated-emotion information Eia, the second-estimated-emotion information Eib, and the third-estimated-emotion information Eic is created. The primary model 100 outputs the primary-estimated-emotion information Ei1 as the primary knowledge information Ki1 to the secondary-biological-information-distillation model 31.

In the secondary-biological-information-distillation model 31, of the electrocardiogram signal Bi1, the pulse wave Bi2, the brain wave Bi3, the sweating Bi4, and the respiration Bi5 that are biological-information-for-learning BiL input to the primary model 100, the electrocardiogram signal Bi1 and the pulse wave Bi2 are input to the convolutional layer 32. The controller 20 creates the multidimensional vectors V related to the electrocardiogram signal Bi1 and the pulse wave Bi2, based on the electrocardiogram signal Bi1 and the pulse wave Bi2 using the convolutional layer 32 of the secondary-biological-information-distillation model 31. The controller 20 outputs the created multidimensional vectors V to the fully connected layer 33. The controller 20 creates the secondary-estimated-emotion information Ei2, based on the multidimensional vectors V, using the fully connected layer 33.

The controller 20 calculates the error L1 of the secondary-estimated-emotion information Ei2 with respect to the emotion-information-for-learning EiL of the training data. Furthermore, the controller 20 calculates the error L2 of the secondary-estimated-emotion information Ei2 with respect to the primary-estimated-emotion information Ei1 that is the primary knowledge information Ki1. The controller 20 adjusts the parameters of the convolutional layer 32 and the fully connected layer 33 of the secondary-biological-information-distillation model 31 such that an overall error L obtained by adding the error L1 and the error L2 together at the arbitrary rate a is as small as possible under the arbitrary ratio α (see Expression 1). At this time, parameters of the encoder sections and the head sections of the learned primary model 100 remain unadjusted.

The primary knowledge information Ki1 is the primary-estimated-emotion information Ei1 as an output of the primary model 100 obtained based on the plurality of different types of the biological-information-for-learning BiL using the primary model 100. That is, the primary knowledge information Ki1 is information (knowledge of the primary model 100) used for estimating the emotion of the user U, obtained by the primary model 100 by learning based on the electrocardiogram signal Bi1, the pulse wave Bi2, the brain wave Bi3, the sweating Bi4 and the respiration Bi5 that are the plurality of types of the biological information Bi including the third biological information Bit.

By using the primary knowledge information Ki1, the secondary-biological-information-distillation model 31 performs learning in a state where the primary-estimated-emotion information Ei1, which includes the brain wave Bi3 and the sweating Bi4 as the second biological information Bis as well as information related to the respiration Bi5 that are information (privileged information) absent from the electrocardiogram signal Bi1 and the pulse wave Bi2 that are the first biological information Bif used for learning of the secondary-biological-information-distillation model 31, is inherited (distillation of knowledge).

As described above, in the secondary-biological-information-distillation model 31 that performed learning using the primary knowledge information Ki1, in addition to the electrocardiogram signal Bi1 and the pulse wave Bi2, emotion estimation accuracy can be increased to a higher level than that when learning was performed based on the electrocardiogram signal Bi1 and the pulse wave Bi2 without using the primary knowledge information Ki1. The secondary-biological-information-distillation model 31 can achieve estimation accuracy close to the emotion estimation accuracy of the primary model 100 even when learning is performed based on the electrocardiogram signal Bi1 and the pulse wave Bi2 that are two types of biological information, that is, fewer types of biological information than five types of biological information, that is, the electrocardiogram signal Bi1, the pulse wave Bi2, the brain wave Bi3, the sweating Bi4, and the respiration Bi5, used by the primary model 100 in obtaining the primary knowledge information Ki1.

The primary model 100 includes the first internal model 100a including the first encoder section 111 and the first head section 121, the second internal model 100b including the second encoder section 112 and the second head section 122, and the third internal model 100c including the third encoder section 113 and the third head section 123 that are used for estimating the emotion, based on the plurality of different types of biological-information-for-learning BiL. In the primary model 100, the primary knowledge information Ki1, in which error and variation in the primary-estimated-emotion information Ei1 are suppressed, is created by ensemble learning, which averages outputs of the first internal model 100a, the second internal model 100b, and the third internal model 100c, each of which receives the plurality of different types of biological-information-for-learning BiL.

The secondary-biological-information-distillation model 31 performs learning, based on the primary knowledge information Ki1 including information about the electrocardiogram signal Bi1, the pulse wave Bi2, the brain wave Bi3, the sweating Bi4, and the respiration Bi5, in addition to the electrocardiogram signal Bi1 and the pulse wave Bi2. Therefore, the secondary-biological-information-distillation model 31 performs learning using the primary knowledge information Ki1 that is an output of the primary model 100 in which error and variation in the emotion estimation accuracy are suppressed. Thus, by estimating the emotion, based on the electrocardiogram signal Bi1 and the pulse wave Bi2 that can be easily acquired using the sensor 11, emotion estimation accuracy can be increased while convenience in acquiring the biological information Bi is increased.

As illustrated in FIG. 3, in estimating the emotion of the user U, the controller 20 of the emotion estimation device 1 estimates the emotion of the user U, based on the biological information related to the electrocardiogram signal Bi1 or the pulse wave Bi2 of the user U, using the secondary-biological-information-distillation model 31. The secondary-biological-information-distillation model 31 has learned the primary knowledge information Ki1 (see FIG. 7) obtained by the primary model 100 by learning. Therefore, the controller 20 can estimate the emotion of the user U, based on the electrocardiogram signal Bi1 or the pulse wave Bi2 that is the first biological information Bif that can be easily measured by the wearable sensor, such as, for example, a smartwatch, a band-type-heart-rate-sensor, or the like (see FIG. 8), among the biological information Bi of the user U, using the secondary-biological-information-distillation model 31. That is, the controller 20 estimates the emotion of the user U, based on the biological information Bi of the user U that is free of the second biological information Bis that is difficult to measure by the wearable sensor or the like, using the secondary-biological-information-distillation model 31, and outputs the secondary-estimated-emotion information Ei2.

The controller 20 estimates the emotion of the user U, based on the biological information Bi related to the electrocardiogram signal Bi1 or the pulse wave Bi2 of the user U that is the biological information Bi the number of types of which is smaller than the electrocardiogram signal Bi1, the pulse wave Bi2, and the respiration Bi5 included in the third biological information Bit and which includes a portion of the electrocardiogram signal Bi1 or the pulse wave Bi2, using the secondary-biological-information-distillation model 31. The controller 20 can estimate the emotion of the user U, using the secondary-biological-information-distillation model 31, based on the electrocardiogram signal Bi1 or the pulse wave Bi2 that can be easily measured by the wearable sensor, such as, for example, a smartwatch, a band-type-heart-rate-sensor, or the like (see FIG. 8) among the biological-information-for-learning BiL used for learning of the secondary-biological-information-distillation model 31. That is, the controller 20 estimates the emotion of the user U, using the secondary-biological-information-distillation model 31, based on the biological information Bi that is a portion of the third biological information Bit and the number of types of which is small among the plurality of types of the biological-information-for-learning BiL including the third biological information Bit used when learning of the secondary-biological-information-distillation model 31 is performed, and outputs the secondary-estimated-emotion information Ei2.

The emotion estimation device 1 can estimate the emotion without limiting at least one of the surrounding environment of the user U, the action of the user U, or the work of the user U, by using the electrocardiogram signal Bi1 and the pulse wave Bi2 of the user U that are the first biological information Bif including at least one of the biological information Bi that is less likely to be affected by the surrounding environment of the user U, the work of the user U, the action of the user U, or the like, the biological information Bi that can be easily acquired from the user U by the wearable sensor or the like, or the biological information Bi that is less likely to be affected by the surrounding environment of the user U, the work of the user U, the action of the user U, or the like and can be easily acquired from the user U by the wearable sensor or the like. The emotion estimation device 1 can easily acquire the electrocardiogram signal Bi1 and the pulse wave Bi2 of the user U performing a specific work, action, or the like, such as, for example, the user U driving a four-wheel automobile and a motorcycle or operating a vessel, an airplane, various types of work machines, a drone, or the like, the walking user U, the user U during exercise such as running, the user U watching a movie, appreciating a picture, viewing contents, or the like, by the wearable sensor or the like.

The emotion estimation device 1 can estimate the emotion without limiting at least one of the surrounding environment of the user U, the action of the user U, or the work of the user U, based on the primary knowledge information Ki1 that includes information related to the brain wave Bi3 and the sweating Bi4 of the user U that are the second biological information Bis including at least one of the biological information Bi that is likely to be affected by the surrounding environment of the user U, the work of the user U, the action of the user U, or the like, the biological information Bi that is difficult to acquire easily from the user U by the wearable sensor or the like, or the biological information Bi that is likely to be affected by the surrounding environment of the user U, the work of the user U, the action of the user U, or the like and is difficult to acquire easily from the user U by the wearable sensor or the like, as well as the biological information Bi of the user U that is free of the second biological information Bis.

The secondary-biological-information-distillation model 31 is created using the primary knowledge information Ki1 including information related to the brain wave Bi3 and the sweating Bi4 that are second biological information Bis, and therefore, has higher estimation accuracy than that of a model that estimates the emotion, based on only the electrocardiogram signal Bi1 or the pulse wave Bi2 of the user U. Thus, by using the biological information Bi that can be easily acquired from the user U for emotion estimation, emotion estimation accuracy can be increased while convenience in acquiring the biological information Bi in estimating the emotion is increased.

The emotion estimation device 1 that estimates the emotion, based on the electrocardiogram signal Bi1 and the pulse wave Bi2 of the user U and the primary knowledge information Ki1 acquires a smaller amount of biological information than that when estimating the emotion of the user U, based on the plurality of different types of the biological-information-for-learning BiL, such as the electrocardiogram signal Bi1, the pulse wave Bi2, the brain wave Bi3, the sweating Bi4, the respiration Bi5, or the like. Therefore, the emotion estimation device 1 can estimate the emotion of the user U with estimation accuracy close to the emotion estimation accuracy of the primary model 100 by using the primary knowledge information Ki1 while suppressing increase in processing load of a hardware.

### Third Embodiment

### <Emotion Estimation Based on Electrocardiogram Information or Pulse Wave>

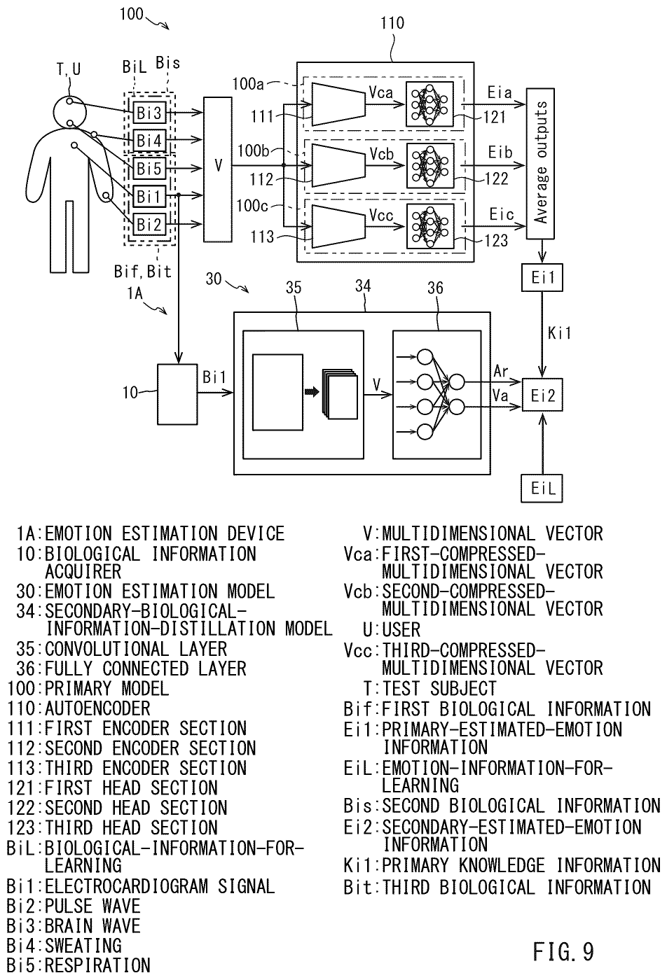

With reference to FIG. 9, an overall configuration of an emotion estimation device 1A according to a third embodiment of the present teaching will be described. FIG. 9 illustrates a configuration when learning of a secondary-biological-information-distillation model 34 included in an emotion estimation model 30 in the emotion estimation device 1A according to the third embodiment of the present teaching is performed using an output of a primary model 100.

As illustrated in FIG. 9, the emotion estimation device 1A creates emotion information Ei related to an emotion of a user U, based on biological information Bi related to an electrocardiogram signal Bi1 of the user U, using the emotion estimation model 30 of a controller 20.

A biological information acquirer 10 of the emotion estimation device 1A acquires the electrocardiogram signal Bi1 or a pulse wave Bi2 that is the biological information Bi related to heart rate of the user U among the biological information Bi using a sensor 11. In this embodiment, the emotion estimation device 1A acquires the electrocardiogram signal Bi1. The biological information acquirer 10 is configured to acquire, for example, the electrocardiogram signal Bi1 of the user U for every unit time using a wearable sensor that can be easily worn by the user U.

The emotion estimation model 30 of the controller 20 is a neural network model that has learned based on training data that is a combination of biological-information-for-learning BiL related to an electrocardiogram signal Bi1 of a test subject T and emotion-information-for-learning EiL based on self-assessment of the test subject T. The emotion estimation model 30 is configured such that the electrocardiogram signal Bi1 of the user U acquired by the biological information acquirer 10 can be input thereto.

### <Creation of Secondary-Biological-Information-Distillation Model 34 (Emotion Estimation Model 30)>

Next, creation of the emotion estimation model 30 will be described. The emotion estimation model 30 includes the secondary-biological-information-distillation model 34 created by learning primary knowledge information Ki1 obtained based on an electrocardiogram signal Bi1, a pulse wave Bi2, a brain wave Bi3, a sweating Bi4, and a respiration Bi5 that are a plurality of different types of the biological-information-for-learning BiL using the learned primary model 100. The secondary-biological-information-distillation model 34 includes a convolutional layer 35 used for calculating a multidimensional vector V of the electrocardiogram signal Bi1 and a fully connected layer 36 used for calculating a secondary-estimated-emotion information Ei2 including the emotional arousal Ar and the valence value Va of an emotion estimated based on the multidimensional vector V. The secondary-biological-information-distillation model 34 outputs the secondary-estimated-emotion information Ei2 obtained by estimating the emotion of the user U with respect to the electrocardiogram signal Bi1 included in first biological information Bif.

In the secondary-biological-information-distillation model 34, the electrocardiogram signal Bi1 is input to the convolutional layer 35. The convolutional layer 35 is used for creating the multidimensional vector V based on the electrocardiogram signal Bi1. The secondary-biological-information-distillation model 34 outputs the multidimensional vector V to the fully connected layer 36. The fully connected layer 36 is used for creating secondary-estimated-emotion information Ei2, based on the multidimensional vector V.

The controller 20 calculates an error L1 of the secondary-estimated-emotion information Ei2 with respect to the emotion-information-for-learning EiL of the training data. Furthermore, the controller 20 calculates an error L2 of the secondary-estimated-emotion information Ei2 with respect to primary-estimated-emotion information Ei1 that is the primary knowledge information Ki1. The controller 20 adjusts parameters of the convolutional layer 35 and the fully connected layer 36 such that an overall error L obtained by adding the error L1 and the error L2 together at the arbitrary rate a is as small as possible (see Expression 1).

The secondary-biological-information-distillation model 34 that has performed learning using the primary knowledge information Ki1 in the above-described manner can inherit information that is absent from the electrocardiogram signal Bi1 and was obtained by the primary model 100 by learning. Therefore, the secondary-biological-information-distillation model 34 has estimation accuracy close to emotion estimation accuracy of the primary model 100, even when learning was performed based on the electrocardiogram signal Bi1 that is biological information a number of types of which is smaller than five types of the biological information, that is, the electrocardiogram signal Bi1, the pulse wave Bi2, the brain wave Bi3, the sweating Bi4, and the respiration Bi5, used in obtaining the primary knowledge information Ki1 using the primary model 100. Thus, by using the electrocardiogram signal Bi1 that can be easily acquired from the user U for estimating the emotion, emotion estimation accuracy can be increased while convenience in obtaining the biological information Bi in estimating the emotion is increased.

### Fourth Embodiment

### <Emotion Estimation Using Tertiary-Biological-Information-Distillation Model>

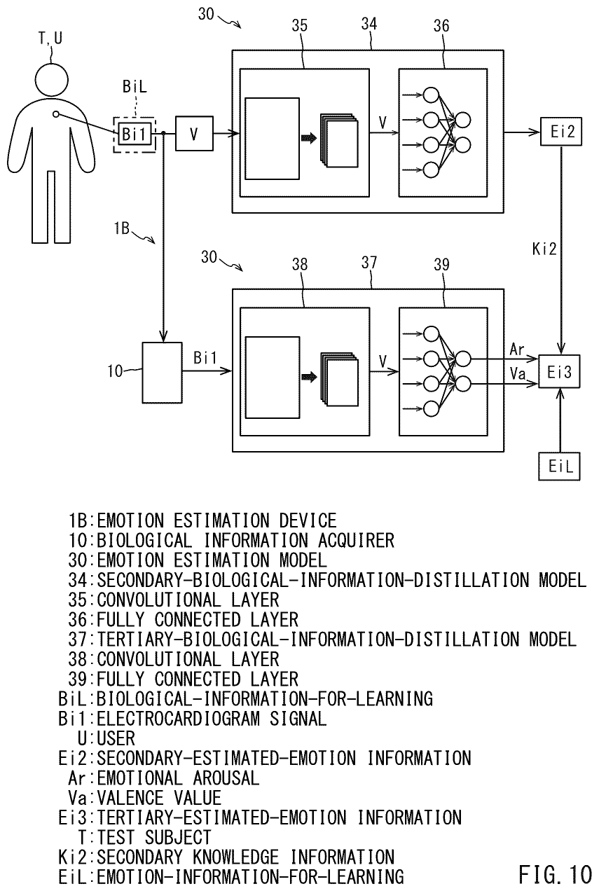

With reference to FIG. 10, an emotion estimation device 1B according to a fourth embodiment of the present teaching will be described. FIG. 10 illustrates a configuration when learning on a tertiary-biological-information-distillation model 37 included in the emotional estimation model 30 in the emotion estimation device 1B according to a fourth embodiment of the present teaching is performed using an output of the secondary-biological-information-distillation model 34.

As illustrated in FIG. 10, the emotion estimation device 1B according to the fourth embodiment of the present teaching creates emotion information Ei related to an emotion of a user U from biological information Bi related to an electrocardiogram signal Bi1 of the user U using the emotion estimation model 30 of the controller 20.

The emotion estimation model 30 of the controller 20 is a neural network model that has performed learning based on training data that is a combination of the electrocardiogram signal Bi1 that is biological-information-for-learning BiL and emotion-information-for-learning EiL based on self-assessment of a test subject T. The emotion estimation model 30 is configured such that the biological-information-for-learning BiL related to the electrocardiogram signal Bi1 of the user U obtained by a sensor 11 can be input thereto.

### <Creation of Tertiary-Biological-Information-Distillation Model>

Next, creation of the emotion estimation model 30 will be described. The emotion estimation model 30 includes the tertiary-biological-information-distillation model 37 created by learning secondary knowledge information Ki2 obtained based on a multidimensional vector V of the biological-information-for-learning BiL using the learned secondary-biological-information-distillation model 34. In this embodiment, the tertiary-biological-information-distillation model 37 performs learning using, as the secondary knowledge information Ki2, the secondary-estimated-emotion information Ei2 created by the learned secondary-biological-information-distillation model 34, based on the multidimensional vector V of the biological-information-for-learning BiL.

The tertiary-biological-information-distillation model 37 is used for estimating an emotion, based on a smaller number of types of the biological-information-for-learning BiL than a number of types of the biological-information-for-learning BiL used in obtaining the secondary knowledge information Ki2 using the secondary-biological-information-distillation model 34. The tertiary-biological-information-distillation model 37 includes a convolutional layer 38 used for calculating the multidimensional vector V of the biological information Bi and a fully connected layer 39 used for calculating tertiary-estimated-emotion information Ei3 including the emotional arousal Ar and the valence value Va of an emotion estimated based on the multidimensional vector V.

The controller 20 adjusts parameters of the convolutional layer 38 and the fully connected layer 39 such that a value obtained by summing an error L1 of the tertiary-estimated-emotion information Ei3 with respect to the emotion-information-for-learning EiL of training data and an error L2 of the tertiary-estimated-emotion information Ei3 with respect to the secondary-estimated-emotion information Ei2 that is the secondary knowledge information Ki2 of the secondary-biological-information-distillation model 34 at an arbitrary rate a is as small as possible (see Expression 1).

The tertiary-biological-information-distillation model 37 configured in the above-described manner performs learning using the secondary-estimated-emotion information Ei2 that is obtained based on the multidimensional vector V of the biological-information-for-learning BiL that is the training data using the learned secondary-biological-information-distillation model 34, the tertiary-estimated-emotion information Ei3 that is obtained based on the biological-information-for-learning BiL using the tertiary-biological-information-distillation model 37, and the emotion-information-for-learning EiL of the training data.

In this embodiment, the secondary-biological-information-distillation model 34 is a model that has performed learning, based on the primary knowledge information Ki1 created using the primary model 100 and electrocardiogram signals Bi1 of the plurality of test subject T (see FIG. 9). In performing learning of the tertiary-biological-information-distillation model 37, the electrocardiogram signal Bi1 that is the biological-information-for-learning BiL that is the training data is used for the secondary-biological-information-distillation model 34. In the tertiary-biological-information-distillation model 37, the electrocardiogram signal Bi1 of the test subject T that is training data is used.

In performing learning on the tertiary-biological-information-distillation model, in the secondary-biological-information-distillation model 34, the electrocardiogram signal Bi1 is input to the convolutional layer 35. In the secondary-biological-information-distillation model 34, the multidimensional vector V related to the electrocardiogram signal Bi1 is created based on the electrocardiogram signal Bi1 using the convolutional layer 35. The secondary-biological-information-distillation model 34 outputs the multidimensional vector V to the fully connected layer 36. In the secondary-biological-information-distillation model 34, secondary-estimated-emotion information Ei2 is created based on the multidimensional vector V using the fully connected layer 36.

In the tertiary-biological-information-distillation model 37, the electrocardiogram signal Bi1 that is the same electrocardiogram signal Bi1 input to the secondary-biological-information-distillation model 34 is input to the convolutional layer 38. In the tertiary-biological-information-distillation model 37, the multidimensional vector Vis created based on the electrocardiogram signal Bi1 using the convolutional layer 38. The tertiary-biological-information-distillation model 37 outputs the multidimensional vector V to the fully connected layer 39. In the tertiary-biological-information-distillation model 37, the tertiary-estimated-emotion information Ei3 is created based on the multidimensional vector V using the fully connected layer 39.

The controller 20 calculates the error L1 of the tertiary-estimated-emotion information Ei3 with respect to the emotion-information-for-learning EiL of the training data. Furthermore, the controller 20 calculates the error L2 of the tertiary-estimated-emotion information Ei3 with respect to the secondary-estimated-emotion information Ei2 that is the secondary knowledge information Ki2. The controller 20 adjusts the parameters of the convolutional layer 38 and the fully connected layer 39 such that the overall error L obtained by adding the error L1 and the error L2 together at the arbitrary rate a is as small as possible. At this time, parameters of an encoder section of the learned secondary-biological-information-distillation model 34 and the fully connected layer remain unadjusted.

The secondary knowledge information Ki2 is information used for estimating the emotion based on the electrocardiogram signal Bi1 of the user U, obtained by the secondary-biological-information-distillation model 34 by learning the electrocardiogram signal Bi1 and the primary knowledge information Ki1. That is, the secondary knowledge information Ki2 includes information (knowledge) obtained by the primary model 100 and the secondary-biological-information-distillation model 34 by learning. The tertiary-biological-information-distillation model 37 that has performed learning using the secondary knowledge information Ki2 can inherit information that is absent from the electrocardiogram signal Bi1.

The tertiary-biological-information-distillation model 37 that has performed learning using the secondary knowledge information Ki2 in the above-described manner can achieve estimation accuracy close to emotion estimation accuracy of the secondary-biological-information-distillation model 34 even when learning was performed based on the electrocardiogram signal Bi1 of the user U that is a smaller number of types of biological information than the number of types of biological-information-for-learning BiL used in obtaining the primary knowledge information Ki1, using the secondary-biological-information-distillation model 31. Thus, by using the biological information Bi that can be easily acquired from the user U for estimating the emotion, emotion estimation accuracy can be increased while convenience in acquiring the biological information Bi in estimating the emotion is increased.

### (Other Embodiments)

Note that, in each of the above-described embodiments, the controller 20 estimates the emotion of the user U or the test subject T using at least one type of the biological information Bi among the electrocardiogram signal Bi1, the pulse wave Bi2, the brain wave Bi3, the sweating Bi4, and the respiration Bi5 of the user U or the test subject T. However, the biological information used by the controller may be some other biological information than the biological information related to the electrocardiogram signal Bi1, the pulse wave Bi2, the brain wave Bi3, the sweating Bi4, and the respiration Bi5. For example, the controller may be configured to estimate the emotion, based on myoelectric potential, body surface temperature, eyeball movement, pupil state, triaxial acceleration of a body, or the like.

In each of the above-described embodiments, the controller 20 creates the emotional arousal Ar and the valence value Va of the emotion that is information related to the emotion of the user U or the test subject T as the secondary-estimated-emotion information Ei2 or the tertiary-estimated-emotion information Ei3. That is, the emotion estimation model 30 indicates the emotion of the user U or the test subject T, based on a combination of the emotional arousal Ar and the valence value Va of the emotion. However, the emotion estimation model may indicate the emotion of the user U or the test subject T as a ratio of each emotion. For example, the emotion estimation model may indicate the emotion of the user U or the test subject T as joy 0.85, enjoyment 0.12, sadness 0.02, anger 0.01, or the like.

In each of the above-described embodiments, the emotion estimation model 30 indicates the valence value Va of the user U or the test subject T by a degree between the positive emotion and the negative emotion and indicates the emotional arousal Ar by a degree between a state where the emotion is activated and a state where the emotion is deactivated. However, the emotion estimation model may classify the valence value Va and the emotional arousal Ar of the user U or the test subject T into two classifications, that is, a high level and a low level, and thus indicate the valence value Va and the emotional arousal Ar in the two classifications.

In each of the above-described embodiments, the primary model 100 performs ensemble learning in which the primary-estimated-emotion information Ei1, which is an average of the first-estimated-emotion information Eia, the second-estimated-emotion information Eib, and the third-estimated-emotion information Eic created using the first internal model 100a, the second internal model 100b, and the third internal model 100c, is created. However, the primary model may perform ensemble learning of some other type. For example, the above-described primary model may perform ensemble learning, in which the second-estimated-emotion information is created based on the first-estimated-emotion information created by the first internal model using the second internal model, and the third-estimated-emotion information is created based on the second-estimated-emotion information using the third internal model.

In each of the above-described embodiments, in the secondary-biological-information-distillation model 31, the primary-estimated-emotion information Ei1 created using the learned primary model 100 is used as the primary knowledge information Ki1. However, the secondary-biological-information-distillation model may perform learning in which a parameter of an intermediate layer of the learned primary model is used as the primary knowledge information. Similarly, the tertiary-biological-information-distillation model may use a parameter of an intermediate layer of the learned secondary-biological-information-distillation model as the secondary knowledge information.

In each of the above-described embodiments, in the primary model 100, the multidimensional vector V of the biological-information-for-learning BiL is input. However, in the primary model, the biological-information-for-learning may be input. In this case, the primary model is configured as a convolutional neural network model including the convolutional layer. In the secondary-biological-information-distillation model 34 and the tertiary-biological-information-distillation model 37, the biological-information-for-learning BiL is input. However, in the secondary-biological-information-distillation model and the tertiary-biological-information-distillation model, the multidimensional vector of the biological-information-for-learning may be input.

In each of the above-described embodiments, the primary model 100 includes the first encoder section 111, the second encoder section 112, and the third encoder section 113. The primary model 100 includes the first head section 121, the second head section 122, and the third head section 123. However, the primary model may not be a model including at least one of an encoder or a head section.

In each of the above-described embodiments, each of the primary model 100, the secondary-biological-information-distillation models 31 and 34, and the tertiary-biological-information-distillation model 37 is configured of a neural network model. However, each of the primary model, the secondary-biological-information-distillation model, and the tertiary-biological-information-distillation model may be configured of a machine learning model, such as a support vector machine (SVM), a decision tree, a k-nearest neighbor algorithm, Naive Bayes, logistic regression, linear regression, nonlinear regression, or stepwise regression.

Note that, in each of the above-described embodiments, each of the emotion estimation devices 1, 1A, and 1B is configured of the portable terminal Pt, such as a smartphone, owned by the user U and the wearable sensor or the like. Each of the emotion estimation devices 1, 1A, and 1B estimates the emotion of the user U, based on the biological information Bi acquired by the wearable sensor or the like, using the processor and the memory of the portable terminal Pt. However, the portable terminal processor may be configured to output the biological information of the user U stored in the portable terminal memory to the emotion estimation device that is outside the portable terminal Pt. The emotion estimation device may be configured as a portion of the emotion estimation system in which a plurality of emotion estimation devices are coupled to a server via the Internet or the like. In the emotion estimation system, a plurality of emotion estimation devices are coupled via the Internet or the like. The emotion estimation system may be configured to be capable of collecting respective pieces of estimated emotion information of users U from the plurality of emotion estimation devices.

Note that, in each of the above-described embodiments, the emotion estimation model 30 includes the secondary-biological-information-distillation model 31 that has learned the primary knowledge information Ki1 created using the learned primary model 100, or the tertiary-biological-information-distillation model 37 that has learned the secondary knowledge information Ki2 created using the learned secondary-biological-information-distillation model 34. However, the emotion estimation model may include a quaternary model that has learned the tertiary knowledge information with information created using the learned tertiary-biological-information-distillation model used as the tertiary knowledge information. That is, the emotion estimation model may include a model that has learned information created using a learned model. The emotion estimation model may include some other model, program, or the like than a model that estimates an emotion.

Note that, in each of the above-described embodiments, nth-order-biological-information-distillation models are configured as the secondary-biological-information-distillation model 31, the secondary-biological-information-distillation model 34, and the tertiary-biological-information-distillation model 37. The nth-order-biological-information-distillation models may include a quaternary-biological-information-distillation model and a higher-order-biological-information-distillation model than the quaternary-biological-information-distillation model.

Embodiments of the present teaching have been described above, but the above-described embodiments are merely illustrative examples for carrying out the present teaching. Therefore, the present teaching is not limited to the above-described embodiments and the above-described embodiments can be appropriately modified and implemented without departing from the gist of the present teaching.

### REFERENCE SIGNS LIST

1, 1A, 1B Emotion estimation device
10 Biological information acquirer
11 Sensor
12 Communication-device-for-sensor
20 Controller
21 Communication-device-for-controller
30 Emotion estimation model
31, 34 Secondary-biological-information-distillation model
32, 35 Convolutional layer
33, 36 Fully connected layer
37 Tertiary-biological-information-distillation model
38 Convolutional layer
39 Fully connected layer
40 Storage device
50 Output device
Bi Biological information
BiL Biological-information-for-learning
Bi1 Electrocardiogram signal
Bi2 Pulse wave
Bi3 Brain wave
Bi4 Sweating
Bi5 Respiration
Bif First biological information
Bis Second biological information
Bit Third biological information
EiL Emotion-information-for-learning
Ei1 Primary-estimated-emotion information
Ei2 Secondary-estimated-emotion information
Ei3 Tertiary-estimated-emotion information
Eia First-estimated-emotion information
Eib Second-estimated-emotion information
Eic Third-estimated-emotion information
V Multidimensional vector
Vca First-compressed-multidimensional vector
Vcb Second-compressed-multidimensional vector
Vcc Third-compressed-multidimensional vector
Vra First-restored-multidimensional vector
Vrb Second-restored-multidimensional vector
Vrc Third-restored-multidimensional vector
Ki1 Primary knowledge information
Ki2 Secondary knowledge information
100 Primary model
100a First internal model
100b Second internal model
100c Third internal model
110 Autoencoder
111 First encoder section
112 Second encoder section
113 Third encoder section
114 First decoder section
115 Second decoder section
116 Third decoder section
121 First head section
122 Second head section
123 Third head section
L, L1, L2 Error
Pt Portable terminal

1. An emotion estimation device for estimating an emotion of a user, comprising:
a storage device that stores biological information of the user; and
a controller including a processor, the controller being configured to:
acquire the biological information of the user to thereby store the biological information in the storage device, the biological information including a plurality of types,
obtain an emotion estimation model used for estimating an emotion based on the biological information,
estimate the emotion of the user, based on the biological information of the user stored in the storage device, using the emotion estimation model, and
output information related to the estimated emotion as emotion information, wherein
the emotion estimation model includes a total number m of models, m being an integer of 2 or larger, that are:
a primary model, which is used for estimating the emotion based on the plurality of types of the biological information, and generates first-order knowledge information, and
a nth-order-biological-information-distillation model that generates a nth-order knowledge information, n being each integer between 2 and m, the nth-order-biological-information-distillation model being created based on a (n−1)th-order knowledge information; and
the controller estimates the emotion of the user using the nth-order-biological-information-distillation model, based on a subset of the plurality of types of the biological information of the user that are used in the emotion estimation by the primary model, and outputs the information related to the estimated emotion as the emotion information.

2. The emotion estimation device according to claim 1, wherein
the nth-order-biological-information-distillation model is created
using the first-order knowledge information obtained by the primary model created based on the plurality of types of the biological information, the biological information including first biological information that is at least one of
the biological information that is independent of a surrounding environment of the user, a work of the user, or an action of the user,
the biological information that can be easily acquired from the user, or
the biological information that is independent of the surrounding environment of the user, the work of the user, or the action of the user and can be easily acquired from the user or
using the (n−1)th-order knowledge information obtained by the (n−1)th-order-biological-information-distillation model created based on the biological information including the first biological information, and
the controller estimates the emotion of the user, based on the first biological information, using the nth-order-biological-information-distillation model.

3. The emotion estimation device according to claim 1, wherein
the nth-order-biological-information-distillation model is created
using the first-order knowledge information obtained by the primary model created based on the plurality of types of the biological information, the biological information including second biological information that is at least one of
the biological information that is dependent on a surrounding environment of the user, a work of the user, or an action of the user,
the biological information that is difficult to acquire easily from the user, or
the biological information that is dependent on the surrounding environment of the user, the work of the user or the action of the user and is difficult to acquire easily from the user, or
using the (n−1)th-order knowledge information obtained by the (n−1)th-order-biological-information-distillation model created based on the biological information including the second biological information, and
the controller estimates the emotion of the user, based on biological information of the user that is free of the second biological information, using the nth-order-biological-information-distillation model.

4. The emotion estimation device according to claim 1, wherein
the nth-order-biological-information-distillation model is created
using the first-order knowledge information obtained by the primary model created based on the plurality of types of the biological information, the biological information including third biological information that is at least two of a brain wave, sweating, respiration, a pulse wave, or an electrocardiogram signal, or
using the (n−1)th-order knowledge information obtained by the (n−1)th-order-biological-information-distillation model created based on the biological information including the third biological information, and
the controller estimates the emotion of the user, based on the biological information, using the nth-order-biological-information-distillation model, the biological information including a subset of types of the biological information included in the third biological information and a portion of each biological information constituting the subset.

5. The emotion estimation device according to claim 1, wherein
among biological information related to a brain wave, sweating, respiration, an electrocardiogram signal, or a pulse wave of the user, the controller estimates the emotion of the user based on the biological information related to at least one of the electrocardiogram signal, the respiration, or the pulse wave using the emotion estimation model.

6. The emotion estimation device according to claim 1, wherein
the (n−1)th-order-biological-information-distillation model includes a plurality of internal models used for estimating the emotion, based on the plurality of types of the biological information, and
the nth-order-biological-information-distillation model is created using the (n−1)th-order knowledge information obtained based on the plurality of types of the biological information, using the plurality of internal models.

7. The emotion estimation device according to claim 1, wherein
the emotion estimation model includes the nth-order-biological-information-distillation model created using the (n−1)th-order knowledge information that is obtained using the (n−1)th-order-biological-information-distillation model based on, among biological information related to a brain wave, sweating, respiration, a pulse wave or an electrocardiogram signal, a plurality of types of the biological information including the biological information related to at least one of the electrocardiogram signal, the respiration or the pulse wave, and the biological information including the biological information related to at least one of the electrocardiogram signal, the respiration or the pulse wave.

8. The emotion estimation device according to claim 1, wherein
each of the nth-order-biological-information-distillation model and the (n−1)th-order-biological-information-distillation model is a neural network model.

9. A portable terminal that acquires biological information of a user used for estimating the emotion of the user in the emotion estimation device according to claim 1, the portable terminal comprising:
a portable terminal memory that stores the acquired biological information of the user; and
a portable terminal processor, wherein
the portable terminal processor acquires the biological information of the user to store the biological information in the portable terminal memory, and outputs the biological information of the user stored in the portable terminal memory.

10. The portable terminal according to claim 9, wherein
the portable terminal is configured to be communicable with outside, and
the portable terminal processor outputs the biological information of the user stored in the portable terminal memory to the emotion estimation device that is outside the portable terminal.

11. The emotion estimation device according to claim 1, wherein
the emotion estimation device is configured to be communicable with outside, and
the controller acquires the biological information from a portable terminal.

12. The emotion estimation device according to claim 1, wherein the emotion estimation device is a portable terminal.
